(19) United States
(12) Patent Application Publication
(10) Pub. No.: US 2024/0339207 A1
(43) Pub. Date: Oct. 10, 2024

(54) System, Method and Computer Readable Medium for Determining Characteristics Of Surgical Related Items and Procedure Related Items Present for Use in the Perioperative Period

(71) Applicant: University of Virginia Patent Foundation, Charlottesville, VA (US)

(72) Inventors: Matthew J. Meyer, North Garden, VA (US); Tyler Chafitz, Charlottesville, VA (US); Pumoli Malapati, Henrico, VA (US); Nafisa Alamgir, Woodbridge, VA (US); Sonali Luthar, Colonial Heights, VA (US); Gabriele Bright, Moseley, VA (US)

(73) Assignee: University of Virginia Patent Foundation, Charlottesville, VA (US)

(21) Appl. No.: 18/573,382

(22) PCT Filed: Jun. 28, 2022
(86) PCT No.: PCT/US22/35295

§ 371 Date: Dec. 21, 2023

Related U.S. Application Data
(60) Provisional application No. 63/216,285, filed on Jun. 29, 2021.

(51) Int. Cl. G16H 40/40

(52) U.S. Cl. CPC G16H 40/40

(57) ABSTRACT

A system and method to determine characteristics of surgical related items and procedure related items present for use in the perioperative period. The system and method may apply computer vision for determining status and tracking of the surgical related items and procedure related items, as well as related clinical, logistical and operational events in the perioperative period. The system and method provides for an intuitive, automated, and transparent tracking of sterile surgical items (SSI) such as single-use, sterile surgical supplies (SUSSS) and sterile surgical instruments, and quantification of SSI waste. In doing so, the system and method empowers administrators to reduce costs and surgeons to demonstrate usage of important equipment. The system and method removes the guesswork from monitoring and minimizing SSI waste and puts the emphasis on necessity and efficiency.

### CROSS REFERENCE TO RELATED APPLICATIONS

The present application claims benefit of priority under 35 U.S.C § 119 (e) from U.S. Provisional Application Ser. No. 63/216,285, filed Jun. 29, 2021, entitled “Cyber Visual System and Method to Identify and Reduce Single-Use Sterile Surgical Waste”; the disclosure of which is hereby incorporated by reference herein in its entirety.

### FIELD OF INVENTION

The present disclosure relates generally to determining characteristics of surgical related items and procedure related items present for use in the perioperative period. More particularly, the present disclosure relates to applying computer vision for determining status and tracking of the items and related clinical, logistical and operational events in the perioperative period.

### BACKGROUND

One cannot improve what one cannot measure. This is certainly the case for surgical waste in hospitals and ambulatory surgical centers. The huge volume of surgical waste is nearly impossible to track and monitor, and therefore results in massive unnecessary costs, inefficient consumption, and environmental impact.

The United States healthcare industry wastes over $2 billion per day, resulting in more than $750 billion in waste each year. This accounts for roughly 25 percent of total healthcare expenditures [1]. This waste is generated from overtreatment, pricing failures, administrative complexities, and failure to properly coordinate care. This waste also poses an immeasurable environmental cost along with the financial cost. The operating room (OR) is a major source of material and financial waste.

Due to the understandable desire to minimize potential risk and maximize expediency, operating rooms often have a multitude of single-use, sterile surgical supplies (SUSSS) opened and ready for immediate access. However, this leads to the opening and subsequent disposal of many more items than were needed. In 2017, UCSF Health quantified the financial loss from opened and unused, single-use, sterile surgical supplies from neurosurgical cases at $968 per case [2]. This extrapolated to $2.9 million per year for a single neurosurgical department [2].

Single-use, sterile surgical supplies (SUSSS) represent eight percent of the operating room cost but are one of the only modifiable expenses. Single-use, sterile surgical supplies (SUSSS) are a constant focus of perioperative administrators attempts to reduce costs. However, identifying wasted, SUSSS is time intensive, must be done during the clinically critical period of surgical closing and the administratively critical period of operating room turnover, and involves handling objects contaminated with blood and human tissue—thus it is essentially never done.

Perioperative administrators want and need to reduce single-use, sterile surgical waste (SUSSS). Perioperative administrators want and need to make sterile surgical instrument pans more efficient too. But a simple and scalable pathway does not exist to identify and aggregate the perioperative and intraoperative waste of sterile surgical items like supplies and instruments. As the proportion of our country's elderly population grows, our healthcare consumption and waste will continue to increase. This waste impacts not just the bottom-line, but also the environment, and sustainability is becoming more important to healthcare consumers and health systems brands.

Perioperative administrators are under constant pressure to reduce costs of running the operating rooms. One maneuver perioperative administrators frequently employ is negotiating lower prices with a different manufacturer of SUSSS. Every time that occurs it leads to a near revolt among surgeons who inevitably have issues with the quality of the new SUSSS or the proprietary nuances that have to be re-learned. Perioperative administrators need a way to reduce operating room costs without rankling surgeons and proceduralists who bring patients and revenue to the hospital.

There is therefore a long unfelt need in the art for tracking and reducing waste in hospitals and ambulatory surgical centers as well any other medical settings.

There is therefore a long unfelt need in the art for reducing costs, increasing consumption efficiency, and enhancing environmental impact.

### SUMMARY OF ASPECTS OF EMBODIMENTS OF THE PRESENT INVENTION

An aspect of an embodiment of the present invention system, method or computer readable medium provides, among other things, intuitive, automated, and transparent tracking of sterile surgical items (SSI) such as single-use, sterile surgical supplies (SUSSS) and sterile surgical instruments, and quantification of SSI waste. In doing so, the system and method empowers administrators to reduce costs and surgeons to demonstrate usage of important equipment. An embodiment of the computer vision and artificial intelligence (AI) based system and method removes the guesswork from monitoring and minimizing SSI waste and puts the emphasis on necessity and efficiency.

An aspect of an embodiment of the present invention system, method or computer readable medium provides, among other things, intuitive, automated, and transparent tracking of surgical related items and/or procedure related items present in preoperative, intraoperative, and/or postoperative settings and/or simulated preoperative, intraoperative, and/or postoperative settings and quantification of surgical related items and/or procedure related items.

An aspect of an embodiment of the present invention system, method or computer readable medium addresses, among other things, the single-use, sterile surgical waste generated by opened and unused items with minimal impact upon the workflow of the operating room by using computer vision and deep learning. An aspect of an embodiment of the present invention system, method or computer readable medium addresses, among other things, surgical related items and/or procedure related items waste generated by opened and unused items with minimal impact upon the workflow of the operating room by using computer vision and deep learning. In an embodiment, the computer vision model and supporting software system will be able to quantify wasted supplies, compile this information into a database, and ultimately provide insight to hospital administrators for which items are often wasted. This information is critical to maximizing efficiency and reducing both the financial and environmental burdens of wasted supplies. An aspect of an embodiment of the present invention system, method or computer readable medium provides, among other things, an OR-wide software that can be utilized by hospitals and ambulatory surgical centers for waste-reduction and cost-savings initiatives; giving OR administrators a new (and less contentious) negotiation approach to reduce the expense of single-use, sterile surgical items.

An aspect of an embodiment of the present invention system, method or computer readable medium solves, among other things, perioperative administrators SUSSS cost problems without any impact on surgeons and essentially no impact on operating room workflow. An aspect of an embodiment of the present invention system, method or computer readable medium provides, among other things, computer vision, machine learning, and an unobtrusive camera to aggregate SUSSS usage (or other surgical related items and/or procedure related items) from multiple operating rooms and multiple surgeons. Over time perioperative administrators can identify the SUSSS (or other surgical related items and/or procedure related items) that are opened on the surgical scrub table, never used by the surgeon, and then required to be thrown out or resterilized or refurbished. Perioperative administrators can subsequently use this data provided by aspect of an embodiment of the present invention system, method or computer readable medium to eliminate never used SUSSS (or other surgical related items and/or procedure related items) from being brought to the operating room, and to keep seldom used SUSSS (or other surgical related items and/or procedure related items) unopened but available in the operating room (so if they remain unused they can be re-used rather than thrown out). An aspect of an embodiment of the present invention system, method or computer readable medium gives, among other things, perioperative administrators an avenue to reduce operating costs and surgeons get to continue to use the SUSSS (or other surgical related items and/or procedure related items) they need.

The term “perioperative period” as used herein, means: a) three phases of surgery including preoperative, intraoperative, and postoperative; and b) three phases of other medical procedures (e.g., non-invasive, minimally invasive, or invasive procedures) including pre-procedure, intra-procedure, and post-procedure.

The term “preoperative, intraoperative, and postoperative settings” indicate the setting where the three respective phases of surgery or clinical care take place including preoperative, intraoperative, and postoperative phases. A setting is a particular place or type of surroundings where preoperative, intraoperative, and postoperative activities takes place. A setting may include, but not limited thereto, the following: surroundings, site, location, set, scene, arena, room, or facility. The setting may be a real setting or a virtual setting.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It should be appreciated that any of the components or modules referred to with regards to any of the present invention embodiments discussed herein, may be integrally or separately formed with one another. Further, redundant functions or structures of the components or modules may be implemented. Moreover, the various components may be communicated locally and/or remotely with any user/operator/customer/client or machine/system/computer/processor. Moreover, the various components may be in communication via wireless and/or hardwire or other desirable and available communication means, systems and hardware. Moreover, various components and modules may be substituted with other modules or components that provide similar functions.

It should be appreciated that the device and related components discussed herein may take on all shapes along the entire continual geometric spectrum of manipulation of x, y and z planes to provide and meet the environmental, anatomical, and structural demands and operational requirements. Moreover, locations and alignments of the various components may vary as desired or required.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

An aspect of an embodiment of the present invention provides, among other things, a system configured for determining one or more characteristics of surgical related items and/or procedure related items present at preoperative, intraoperative, and/or postoperative settings and/or simulated preoperative, intraoperative, and/or postoperative settings. The system may comprise: one or more computer processors; and a memory configured to store instructions that are executable by said one or more computer processors, wherein said one or more computer processors are configured to execute the instructions to: receive settings image data corresponding with the preoperative, intraoperative, and/or postoperative settings and/or simulated preoperative, intraoperative, and/or postoperative settings; run a trained computer vision model on the received settings image data to identify and label the surgical related items and/or procedure related items in the preoperative, intraoperative, and/or postoperative settings and/or simulated preoperative, intraoperative, and/or postoperative settings; interpret the surgical related items and/or procedure related items through tracking and analyzing said identified and labeled surgical related items and/or procedure related items in the preoperative, intraoperative, and/or postoperative settings and/or simulated preoperative, intraoperative, and/or postoperative settings, to determine said one or more characteristics of the surgical related items and/or procedure related items; and transmit said one or more determined characteristics to a secondary source. In an embodiment, the one or more computer processors may be configured to execute the instructions to: retrain said trained computer vision model using said received settings image data from the preoperative, intraoperative, and/or postoperative settings and/or simulated preoperative, intraoperative, and/or postoperative settings. In an embodiment, the one or more computer processors may be configured to execute the instructions to: wherein the trained computer vision model is generated on preliminary image data using a machine learning algorithm.

The preliminary image data are image data that is similar to data that will be collected or received regarding the surgical related items and/or procedure related items in the preoperative, intraoperative, and/or postoperative settings and/or simulated preoperative, intraoperative, and/or postoperative settings. The preliminary image data may include three dimensional renderings or representation of surgical related items and/or procedure related items in the preoperative, intraoperative, and/or postoperative settings and/or simulated preoperative, intraoperative, and/or postoperative settings

An aspect of an embodiment of the present invention provides, among other things, a computer-implemented method for determining one or more characteristics of surgical related items and/or procedure related items present at preoperative, intraoperative, and/or postoperative settings and/or simulated preoperative, intraoperative, and/or postoperative settings. The method may comprise: receiving settings image data from the preoperative, intraoperative, and/or postoperative settings and/or simulated preoperative, intraoperative, and/or postoperative settings; running a trained computer vision model on the received settings image data to identify and label the surgical related items and/or procedure related items in the preoperative, intraoperative, and/or postoperative settings and/or simulated preoperative, intraoperative, and/or postoperative settings; interpreting the surgical related items and/or procedure related items through tracking and analyzing said identified and labeled surgical related items and/or procedure related items in the preoperative, intraoperative, and/or postoperative settings and/or simulated preoperative, intraoperative, and/or postoperative settings, to determine said one or more characteristics of the surgical related items and/or procedure related items; and transmitting said one or more determined characteristics to a secondary source. In an embodiment, the method may further comprise retraining the trained computer vision model using the received settings image data from the preoperative, intraoperative, and/or postoperative settings and/or simulated preoperative, intraoperative, and/or postoperative settings. In an embodiment, the trained computer vision model is generated on preliminary image data using a machine learning algorithm.

An aspect of an embodiment of the present invention provides, among other things, a non-transitory computer-readable medium storing instructions that, when executed by one or more processors, cause the one or more processors to perform operations for determining one or more characteristics of surgical related items and/or procedure related items present at preoperative, intraoperative, and/or postoperative settings and/or simulated preoperative, intraoperative, and/or postoperative settings. The non-transitory computer-readable medium storing instructions may comprise: receiving settings image data from the preoperative, intraoperative, and/or postoperative settings and/or simulated preoperative, intraoperative, and/or postoperative settings; running a trained computer vision model on the received settings image data to identify and label the surgical related items and/or procedure related items in the preoperative, intraoperative, and/or postoperative settings and/or simulated preoperative, intraoperative, and/or postoperative settings; interpreting the surgical related items and/or procedure related items through tracking and analyzing said identified and labeled surgical related items and/or procedure related items in the preoperative, intraoperative, and/or postoperative settings and/or simulated preoperative, intraoperative, and/or postoperative settings, to determine said one or more characteristics of the surgical related items and/or procedure related items; and transmitting said one or more determined characteristics to a secondary source. In an embodiment, the non-transitory computer-readable medium of may further comprise: retraining said trained computer vision model using said received settings image data from the preoperative, intraoperative, and/or postoperative settings and/or simulated preoperative, intraoperative, and/or postoperative settings. In an embodiment, the trained computer vision model is generated on preliminary image data using a machine learning algorithm.

The invention itself, together with further objects and attendant advantages, will best be understood by reference to the following detailed description, taken in conjunction with the accompanying drawings.

These and other objects, along with advantages and features of various aspects of embodiments of the invention disclosed herein, will be made more apparent from the description, drawings and claims that follow.

### BRIEF DESCRIPTION OF THE DRAWINGS

The foregoing and other objects, features and advantages of the present invention, as well as the invention itself, will be more fully understood from the following description of preferred embodiments, when read together with the accompanying drawings

The accompanying drawings, which are incorporated into and form a part of the instant specification, illustrate several aspects and embodiments of the present invention and, together with the description herein, serve to explain the principles of the invention. The drawings are provided only for the purpose of illustrating select embodiments of the invention and are not to be construed as limiting the invention.

FIG. 1(A) is a screenshot showing photographic depictions of real operating room scrub tables.

FIG. 1(B) is a screenshot showing photographic depictions of real operating room scrub tables whereby the computer vision model has correctly detected the presence of suctions on the table.

FIG. 2 is a screenshot showing a photographic depiction of an example the computer vision model that correctly detected several items of interest on a mock scrub table.

FIG. 3 is a screenshot showing a graphical representation of the computer vision model's mean average precision (mAP).

FIG. 4 is a screenshot showing the annotation tool Dataloop.ai user interface.

FIG. 5 is a screenshot showing photographic depictions of object detection on different frames within the same video represented in FIGS. 5(A)-5(D), respectively.

FIG. 6 is a block diagram of an exemplary process for determining characteristics of surgical related items and procedure related items, consistent with disclosed embodiments.

FIG. 7 is a block diagram of an exemplary process for determining characteristics of surgical related items and procedure related items, consistent with disclosed embodiments.

FIG. 8 is a block diagram illustrating an example of a machine (or in some embodiments one or more processors or computer systems (e.g., a standalone, client or server computer system, cloud computing, or edge computing)) upon which one or more aspects of embodiments of the present invention can be implemented.

FIG. 9 is a screenshot of a flow diagram of a method for determining one or more characteristics of surgical related items and procedure related items.

FIG. 10 is a screenshot of a flow diagram of a method and table for determining one or more characteristics of surgical related items and procedure related items.

FIGS. 11(A)-(B) is a flow diagram of a method for determining one or more characteristics of surgical related items and procedure related items.

### DETAILED DESCRIPTION OF EXEMPLARY EMBODIMENTS

Referring to an aspect of an embodiment of the present invention system, method or computer readable medium provides, for example, the workflow may begin in the operating room, with the setup of a camera (or cameras) to record the activity of the scrub table throughout the surgery. Once the camera is secured, and the operation begins, the camera will continuously (or non-continuously if specified, desired, or required) take photos of the scrub table from a birds-eye-view multiple times each minute or second (or fraction of seconds or minutes, as well as other frequencies or durations or as desired or required) in regular intervals. After completion of the operation, the recording is stopped. The series of images is then transmitted to the computer (or processor) with trained computer vision software, which uses machine learning to recognize and identify the surgical supplies that can be seen in the images of the scrub table. Based on factors such as leaving the field-of-view, or moving to a different spot on the table, the machine learning program can identify if an item has been interacted with, and thus likely used in the surgical setting. Using the aggregate of data analysed from each photo in the surgery, a list of which items were placed on the scrub table can be determined, and then which of those items remained unused throughout the operation can be determined. Over the course of multiple surgeries, an embodiment of the present invention system, method or computer readable medium can compile this information in order to determine which items are most often opened but unused, which can be sorted by type or procedure or surgeon themselves.

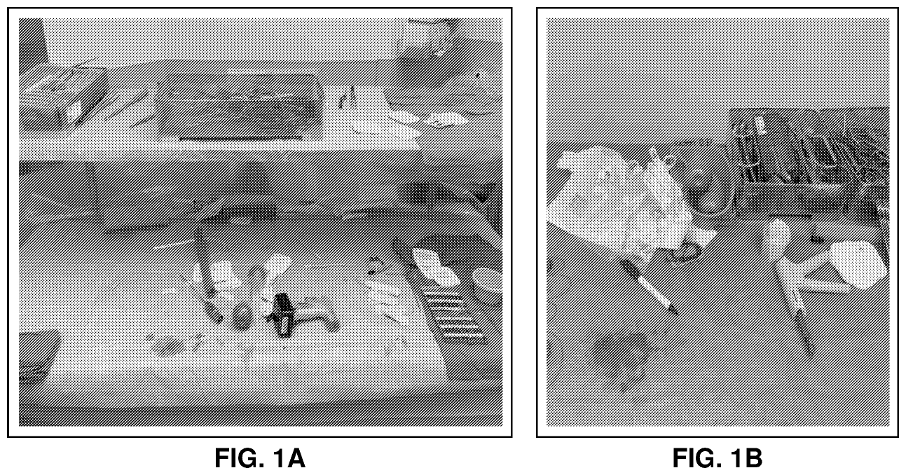

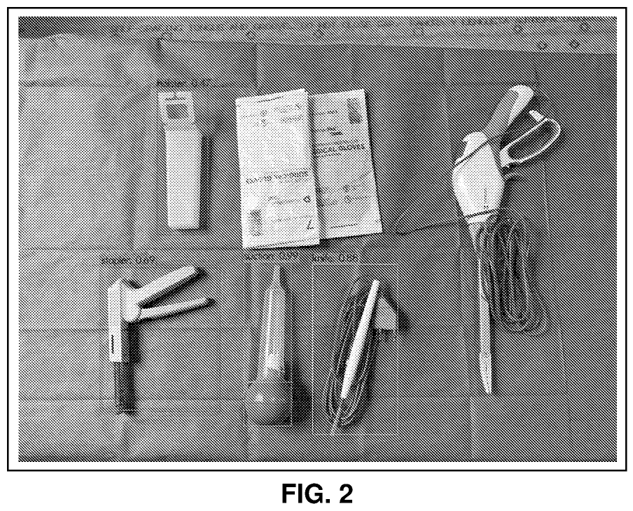

FIG. 1 is a screenshot showing photographic depictions of real operating room scrub tables. There are an assortment of tools and material present after surgery (FIG. 1(A)). In an embodiment of the present invention system, method or computer readable medium the computer vision model has correctly detected the presence of bulb suctions on the table (FIG. 1(B)).

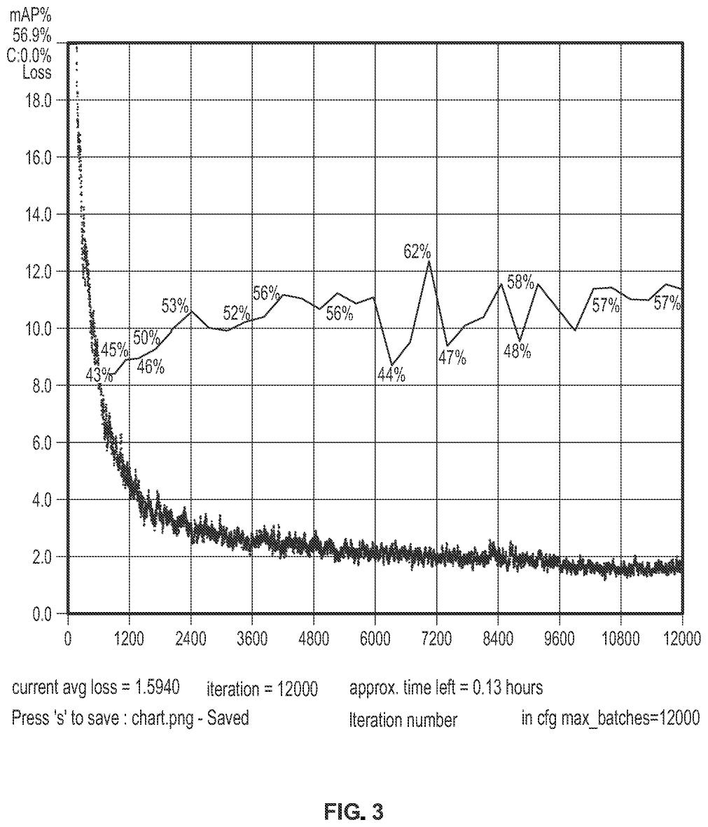

FIG. 2 is a screenshot showing a photographic depiction of an example detection using an embodiment of the present invention system, method or computer readable medium on a previously-unseen image. In an embodiment, the computer vision model correctly detects several items of interest on a mock scrub table. The decimal numbers represent confidence scores of the detection. Other data collection and training may be obtained with other embodiments of the computer vision model.

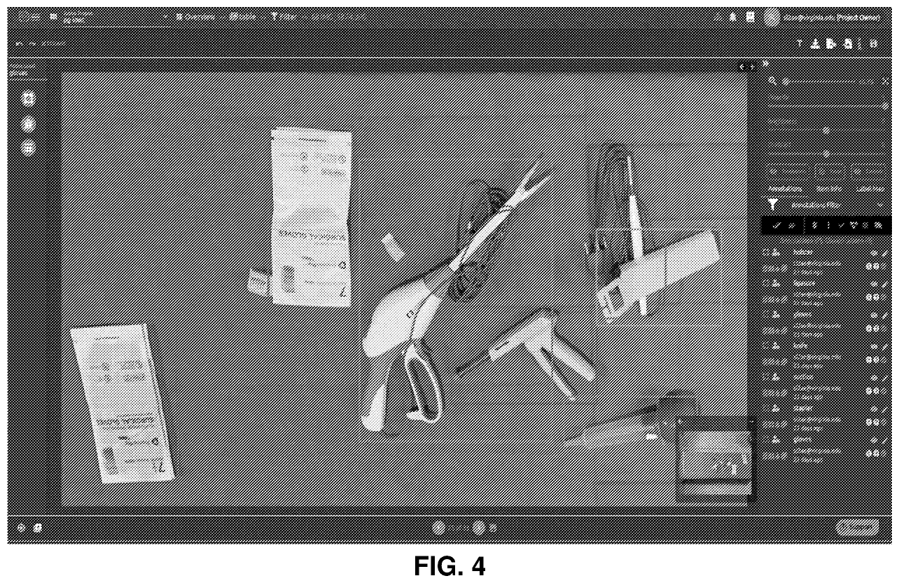

FIG. 3 is a screenshot showing a graphical representation of the computer vision model's mean average precision (mAP) that was created during the training process an embodiment of the present invention system, method or computer readable medium. The mean average precision (mAP) score, shown as the “thin line” on the graph, is a measure of accuracy of the computer vision model and reaches a high of 62 percent. This score is exceptional given that the approach had not yet undertaken advanced techniques to improve the computer vision model detection. Accuracy will likely increase in future iterations. The loss, shown as the “thick line”, decreases as expected as the computer vision model learns over several thousand iterations. Provided below is a formula for the loss used by this specific model (YOLOv4) in an embodiment. However, the formula used to calculate loss in other embodiments will change as we continue to develop the process. Other loss functions as desired or required may be employed in the context of the invention.

$$ℒ ( \hat{z} , z ) = ℒ \text{?} - \text{?} [ \text{?} log ( \text{?} ) + ( 1 - \text{?} ) log ( 1 - \text{?} ) ] - \text{?} [ \text{?} log ( \text{?} ) + ( 1 - \text{?} ) log ( 1 - \text{?} ) ] - \text{?} [ \text{?} ( c ) log ( \text{?} ( c ) ) + ( 1 - \text{?} ( c ) ) log ( 1 - \text{?} ( c ) ) ]$$

$$\text{?} \text{indicates text missing or illegible when filed}$$

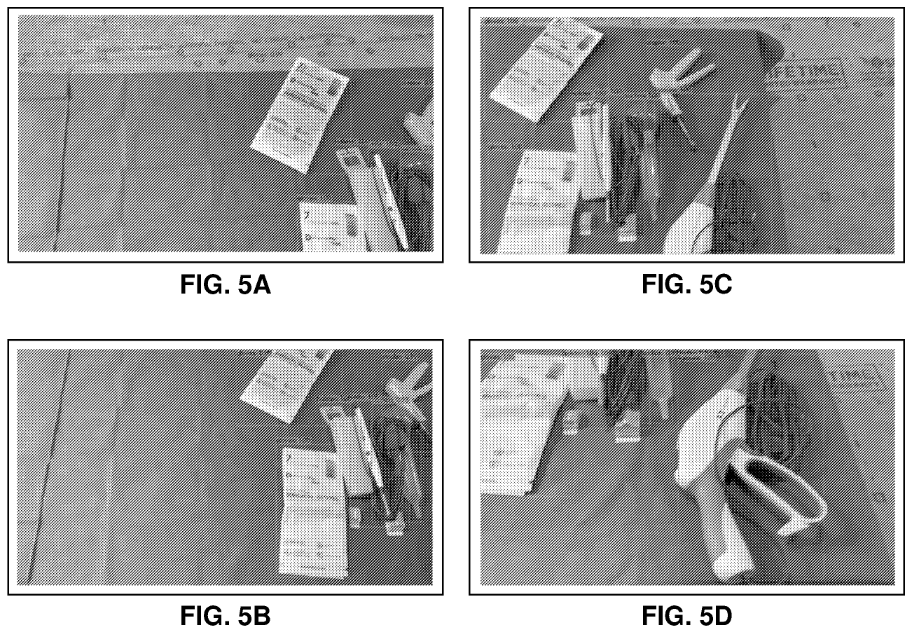

FIG. 4 is a screenshot showing the annotation tool Dataloop.ai user interface. In an embodiment, this tool was used to annotate six objects of interest: gloves, LigaSure, stapler, knife, holster, and suction. This becomes the input data to feed the computer vision model for training. Dataloop.ai's interface serves only as an example of how images are annotated in an embodiment. Other types of interfaces or services for annotations or the like as desired or required may be employed in the context of the invention.

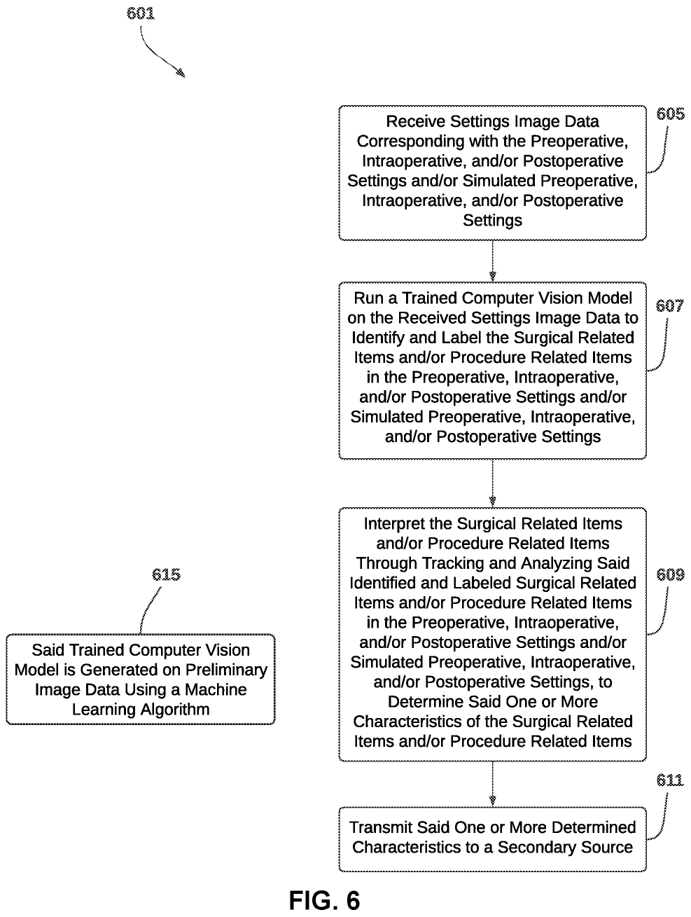

FIG. 5 is a screenshot showing object detection on different frames within the same video represented in FIGS. 5(A)-5(D), respectively, of an embodiment of the present invention system, method or computer readable medium. In an embodiment, the detection only displays objects that are actively visible on the table. In an embodiment, during operation the detection of objects disappear and reappears as the object moves on the camera.

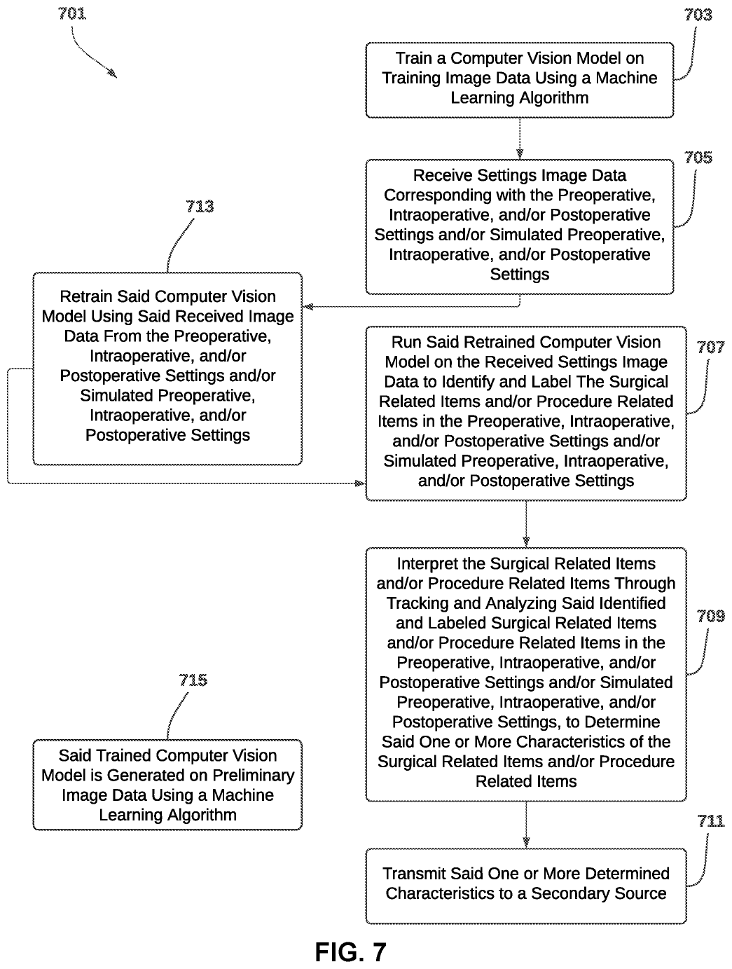

FIG. 6 is a flow diagram of a method 601 for determining one or more characteristics of surgical related items and/or procedure related items present at preoperative, intraoperative, and/or postoperative settings and/or simulated preoperative, intraoperative, and/or postoperative settings. The method 601 can be performed by a system of one or more appropriately-programmed computers or processors in one or more locations.

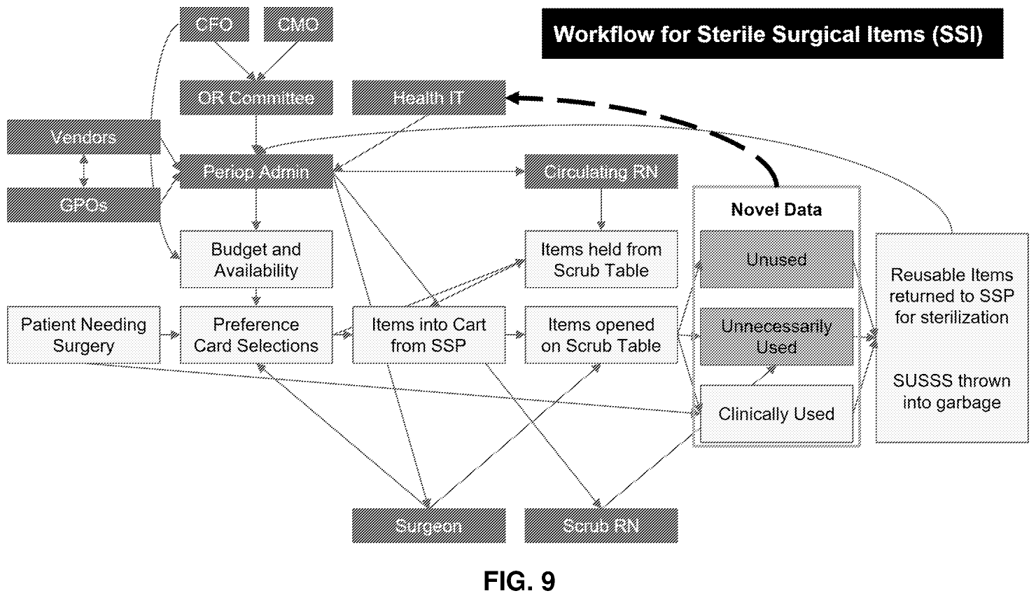

For instance, the flow diagram of an exemplary method for determining one or more characteristics of surgical related items and/or procedure related items present at preoperative, intraoperative, and/or postoperative settings and/or simulated preoperative, intraoperative, and/or postoperative settings, is consistent with disclosed embodiments. The method 601 may be performed by processor 102 of, for example, system 100, which executes instructions 124 encoded on a computer-readable medium storage device (as for example shown in FIG. 8). It is to be understood, however, that one or more steps of the method may be implemented by other components of system 100 (shown or not shown).

Still referring to FIG. 6, at step 605, the system receives settings image data corresponding with the preoperative, intraoperative, and/or postoperative settings and/or simulated preoperative, intraoperative, and/or postoperative settings.

At step 607, the system runs a trained computer vision model on the received settings image data to identify and label the surgical related items and/or procedure related items in the preoperative, intraoperative, and/or postoperative settings and/or simulated preoperative, intraoperative, and/or postoperative settings.

At step 609, the system interprets the surgical related items and/or procedure related items through tracking and analyzing said identified and labeled surgical related items and/or procedure related items in the preoperative, intraoperative, and/or postoperative settings and/or simulated preoperative, intraoperative, and/or postoperative settings, to determine said one or more characteristics of the surgical related items and/or procedure related items.

At step 611, the system transmits said one or more determined characteristics to a secondary source.

In an embodiment, at step 615, the trained computer vision model may be generated on preliminary image data using a machine learning algorithm.

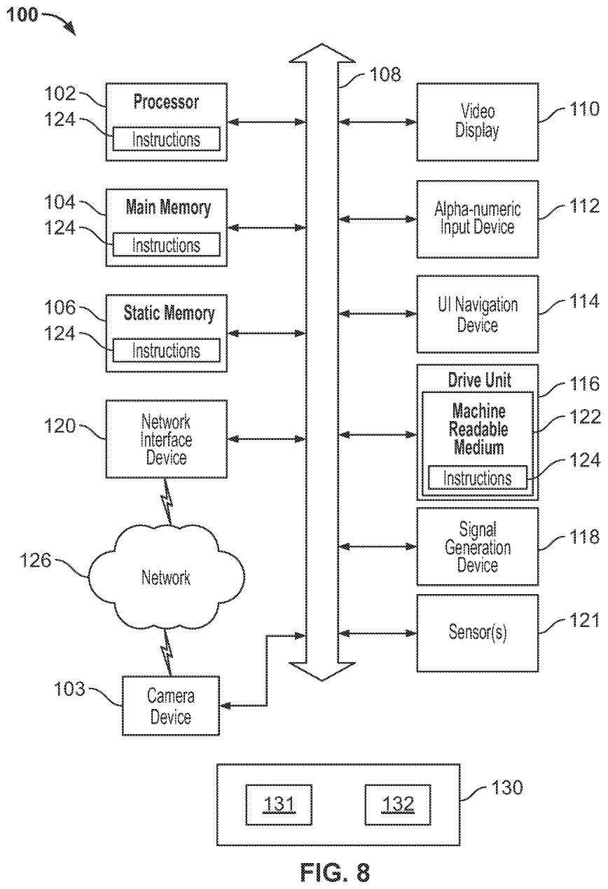

FIG. 7 is a flow diagram of a method 701, similar to the embodiment shown in FIG. 6, for determining one or more characteristics of surgical related items and/or procedure related items present at preoperative, intraoperative, and/or postoperative settings and/or simulated preoperative, intraoperative, and/or postoperative settings. The method 701 can be performed by a system of one or more appropriately-programmed computers or processors in one or more locations.

Still referring to FIG. 7, at step 705, the system receives settings image data corresponding with the preoperative, intraoperative, and/or postoperative settings and/or simulated preoperative, intraoperative, and/or postoperative settings.

At step 713, the system retrains a trained computer vision model using said received settings image data from the preoperative, intraoperative, and/or postoperative settings and/or simulated preoperative, intraoperative, and/or postoperative settings. At step 707, the system runs said retrained computer vision model on the received settings image data to identify and label the surgical related items and/or procedure related items in the preoperative, intraoperative, and/or postoperative settings and/or simulated preoperative, intraoperative, and/or postoperative settings.

At step 709, the system interprets the surgical related items and/or procedure related items through tracking and analyzing said identified and labeled surgical related items and/or procedure related items in the preoperative, intraoperative, and/or postoperative settings and/or simulated preoperative, intraoperative, and/or postoperative settings, to determine said one or more characteristics of the surgical related items and/or procedure related items.

At step 711, the system transmits said one or more determined characteristics to a secondary source.

In an embodiment, at step 715, the trained computer vision model may be generated on preliminary image data using a machine learning algorithm.

Still referring to FIG. 7, in an embodiment, regarding step 713, the system may retrain any number of times as specified, desired, or required. In an embodiment, after the retraining step 713 is complete then the system may perform any one or more of the steps 705, 707, 709, 711, and 715 (and in a variety of sequences or orders as desired or required).

FIG. 8 is a bock diagram of an exemplary system, consistent with disclosed embodiment. FIG. 8 represents an aspect of an embodiment of the present invention that includes, but not limited thereto, a system, method, and computer readable medium that provides for, among other things: determining one or more characteristics of the surgical related items 131 and/or procedure related items 132 present at preoperative, intraoperative, and/or postoperative settings 130 and/or simulated preoperative, intraoperative, and/or postoperative settings 130, which illustrates a block diagram of an example machine 100 (or machines) upon which one or more embodiments (e.g., discussed methodologies) can be implemented (e.g., run). A camera 103 (or cameras) may be provided configured to capture the image of the surgical related items 131 and/or procedure related items 132 present at preoperative, intraoperative, and/or postoperative settings 130 and/or simulated preoperative, intraoperative, and/or postoperative settings 130.

Examples of machine 100 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system, cloud computing, or edge computing) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs).)

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 100) and software architectures that can be deployed in example embodiments.

In an example, the machine 100 can operate as a standalone device or the machine 100 can be connected (e.g., networked) to other machines.

In a networked deployment, the machine 100 can operate in the capacity of either a server or a client machine in server-client network environments. In an example, machine 100 can act as a peer machine in peer-to-peer (or other distributed) network environments. The machine 100 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 100. Further, while only a single machine 100 is illustrated, the term “machine” shall also be taken to include any collection of machines that individually or jointly execute a set (or multiple sets) of instructions to perform any one or more of the methodologies discussed herein.

Example machine (e.g., computer system) 100 can include a processor 102 (e.g., a central processing unit (CPU), a graphics processing unit (GPU) or both), a main memory 104 and a static memory 106, some or all of which can communicate with each other via a bus 108. The machine 100 can further include a display unit 110, an alphanumeric input device 112 (e.g., a keyboard), and a user interface (UI) navigation device 111 (e.g., a mouse). In an example, the display unit 810, input device 417 and UI navigation device 114 can be a touch screen display. The machine 100 can additionally include a storage device (e.g., drive unit) 116, a signal generation device 418 (e.g., a speaker), a network interface device 120, and one or more sensors 121, such as a global positioning system (GPS) sensor, compass, accelerometer, or other sensor.

The storage device 116 can include a machine readable medium 122 on which is stored one or more sets of data structures or instructions 124 (e.g., software) embodying or utilized by any one or more of the methodologies or functions described herein. The instructions 124 can also reside, completely or at least partially, within the main memory 104, within static memory 106, or within the processor 102 during execution thereof by the machine 100. In an example, one or any combination of the processor 102, the main memory 104, the static memory 106, or the storage device 116 can constitute machine readable media.

While the machine readable medium 122 is illustrated as a single medium, the term “machine readable medium” can include a single medium or multiple media (e.g., a centralized or distributed database, and/or associated caches and servers) that configured to store the one or more instructions 124. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 124 can further be transmitted or received over a communications network 126 using a transmission medium via the network interface device 120 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

An aspect of an embodiment of the present invention provides, among other thing, method and related system for determining one or more characteristics of surgical related items and/or procedure related items present at preoperative, intraoperative, and/or postoperative settings and/or simulated preoperative, intraoperative, and/or postoperative settings. The method (and related system) may comprise: receiving settings image data from the preoperative, intraoperative, and/or postoperative settings and/or simulated preoperative, intraoperative, and/or postoperative settings; running a trained computer vision model on the received settings image data to identify and label the surgical related items and/or procedure related items in the preoperative, intraoperative, and/or postoperative settings and/or simulated preoperative, intraoperative, and/or postoperative settings; interpreting the surgical related items and/or procedure related items through tracking and analyzing said identified and labeled surgical related items and/or procedure related items in the preoperative, intraoperative, and/or postoperative settings and/or simulated preoperative, intraoperative, and/or postoperative settings, to determine said one or more characteristics of the surgical related items and/or procedure related items; and transmitting said one or more determined characteristics to a secondary source.

In an embodiment, settings image data may include information from the visible light spectrum and/or invisible light spectrum.

In an embodiment, the settings image data may include three dimensional renderings or representation of information of the surgical related items and/or procedure related items in the preoperative, intraoperative, and/or postoperative settings and/or simulated preoperative, intraoperative, and/or postoperative settings.

In an embodiment, the method (and related system) may also include retraining said trained computer vision model using said received settings image data from the preoperative, intraoperative, and/or postoperative settings and/or simulated preoperative, intraoperative, and/or postoperative settings. In an embodiment, the trained computer vision model may be generated on preliminary image data using a machine learning algorithm.

In some embodiments, the “procedure related item” may include, but not limited thereto, non-invasive, minimally invasive, or invasive instruments, devices., equipment, apparatus, infrastructure, medications/supplies, electronics, monitors, or supplies. The non-invasive instruments vices, equipment, apparatus, infrastructure, medications/supplies, electronics, monitors, or supplies nay be used in a variety of medical procedures, such as, but not limited thereto, cardiovascular, vascular, gastrointestinal, neurological, radiology, pulmonology, and oncology. Other medical procedures as desired or required may be employed in the context of the invention.

In some embodiments, the “surgical related item” may include, but not limited thereto, instruments, devices, equipment, apparatus, infrastructure, medications/supplies, electronics, monitors, or supplies. In some embodiments the infrastructure may include, but not limited thereto the following: intravenous pole, surgical bed, sponge rack, stools, equipment/light boon, or suction canisters, in some embodiments the medications/therapies may include, but not limited thereto the following: vials, ampules, syringes, bags, bottles, tanks (e.g., nitric oxide, oxygen, carbon dioxide), blood products, allografts, or recombinant tissue. In some embodiments the supplies may include, but not limited thereto the following: sponges, trocars, needles, suture, catheters, wires, implants, single-use items, sterile and non-sterile, staplers, staple loads, cautery, or irrigators. In some embodiments the instruments may include, but not limited thereto the following: clamps, needle-drivers, retractors, scissors, scalpel, laparoscopic tools, or reusable and single-use. In some embodiments the electronics may include, but not limited thereto the following: electrocautery, robotic assistance, microscope, laparoscope, endoscope, bronchoscope, tourniquet, ultrasounds, or screens. In some embodiments the resuscitation equipment may include, but not limited thereto the following: defibrillator, code cart, difficult airway cart, video laryngoscope, cell-saver, cardiopulmonary bypass, extracorporeal membrane oxygenation, or cooler for blood products or organ. In some embodiments the monitors may include, but not limited thereto the following: EKG leads, blood pressure cuff, neurostimulators, bladder catheter, or oxygen saturation monitor.

In an embodiment, the method (and related system) may include wherein: said training of said computer vision model, may be performed with one or more of the following configurations: i) streaming to the cloud and in real-time, ii) streaming to the cloud and in delayed time, iii) aggregated and delayed, iv) locally on an edge-computing node, and v) locally and/or remotely on a network and/or server.

In an embodiment, the method (and related system) may include wherein: said retraining of said computer vision model, may be performed with one or more of the following configurations: i) streaming to the cloud and in real-time, ii) streaming to the cloud and in delayed time, iii) aggregated and delayed, iv) locally on an edge-computing node, and v) locally and/or remotely on a network and/or server.

In an embodiment, the method (and related system) may include one or more of the following actions: a) said receiving of said settings image data, b) said running of said trained computer vision model, and c) said interpreting of the surgical related items and/or procedure related items, that may be performed with one or more of the following actions: i) streaming to the cloud and in real-time, ii) streaming to the cloud and in delayed time, iii) aggregated and delayed, iv) locally on an edge-computing node, and v) locally and/or remotely on a network and/or server.

In an embodiment, the method (and related system) of tracking and analyzing may include one or more of the following: object identification for tracking and analyzing; motion sensing for tracking and analyzing; and infrared sensing for tracking and analyzing.

In an embodiment, the method (and related system) of said tracking and analyzing may include specified multiple tracking and analyzing models.

In an embodiment, the method (and related system) for said tracking and analyzing may be performed with one or more of the following: one or more databases; cloud infrastructure; and edge-computing;

In an embodiment, the method (and related system) wherein said secondary source includes one or more of any one of the following: local memory; remote memory; or display or graphical user interface.

In an embodiment, the method (and related system) wherein the machine learning algorithm includes an artificial neural network (ANN) or deep learning algorithm.

In an embodiment, the method (and related system) wherein said artificial neural network (ANN) includes: convolutional neural network (CNN); and/or recurrent neural networks (RNN).

In an embodiment, the method (and related system) wherein said determined one or more characteristics includes any combination of one or more of the following: identification of the one or more of the surgical related items and/or procedure related items; usage or non-usage status of the one or more of the surgical related items and/or procedure related items; opened or unopened status of the one or more of the surgical related items and/or procedure related items; moved or non-moved status of the one or more of the surgical related items and/or procedure related items; single-use or reusable status of the one or more of the surgical related items and/or procedure related items; or association of clinical events, logistical events, or operational events.

In an embodiment, the method (and related system) may include one or more cameras configured to capture the image to provide said received image data.

In some embodiments, the camera may be configured to operate in the visible spectrum as well as the invisible spectrum. The visible spectrum, sometimes referred to as the optical spectrum or luminous spectrum, is that portion of the electromagnetic spectrum that is visible to (i.e., can be detected by) the human eye and may be referred to as visible light or simply light. A typical human eye will respond to wavelengths in air that are from about 380 nm to about 750 nm. The invisible spectrum (i.e., the non-luminous spectrum) is that portion of the electromagnetic spectrum that lies below and above the visible spectrum (i.e., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In an embodiment, the method (and related system) wherein based on said determined one or more characteristics, may further include: determining an actionable output to reduce unnecessary waste of the surgical related items for use in the preoperative, intraoperative, and/or postoperative settings (e.g., guiding sterile kits of the surgical related items) and/or the simulated preoperative, intraoperative phase, and/or postoperative settings; determining an actionable output to reorganize the surgical related items for use in the preoperative, intraoperative, and/or postoperative settings or the simulated preoperative, intraoperative, and/or postoperative settings; determining an actionable output to reduce supply, storage, sterilization and disposal costs associated with use of the surgical related items for use in the preoperative, intraoperative, and/or postoperative settings and/or the simulated preoperative, intraoperative, and/or postoperative settings; determining an actionable output to reduce garbage and unnecessary re-sterilization associated with use of the surgical related items for use in the preoperative, intraoperative, and/or postoperative settings and/or the simulated preoperative, intraoperative, and/or postoperative settings; determining an actionable output to streamline setup of the surgical related items for use in the preoperative, intraoperative, and/or postoperative settings and/or the simulated preoperative, intraoperative, and/or postoperative settings; determining an actionable output to improve efficiency of using the surgical related items for use in the preoperative, intraoperative, and/or postoperative settings and/or the simulated preoperative, intraoperative, and/or postoperative settings; determining an actionable output to identify, rank, and/or recognize level of efficiency of surgeons or clinicians; and/or determining an actionable output to improve the level of efficiency of using the surgical related items and/or procedure related items that are sterilized.

In an embodiment, the method (and related system) does not require a) machine readable markings on the surgical related items and/or procedure related items nor b) communicable coupling between said surgical related items and/or procedure related items and the system (and related method) to provide said one or more determined characteristics. Examples of the machine readable markings may include, but not limited thereto, the following: a RFID sensor; a UPC, EAN or GTIN; an alpha-numeric sequential marking; and/or an easy coding scheme that is readily identifiable by a human for redundant checking purposes.

In an embodiment, consistent identification of the identified and labeled surgical related items and/or procedure related items in the preoperative, intraoperative, and/or postoperative settings and/or simulated preoperative, intraoperative, and/or postoperative settings, may include the following standard: mean average precision greater than 90 percent. In some embodiments, the standard of the mean average precision may be specified to be greater than or less than 90 percent. The following formula for mean average precision (mAP) is provided below. The mean average precision is the mean average precisions for all classes (as in, the average precisions with which the model detects the presence of each type of object in images).

$$\begin{matrix}mAP = \frac{1}{n} {\sum }_{i ϵ n} \frac{\vert {TP}_{i} \vert}{\vert {FP}_{i} \vert + \vert {TP}_{i} \vert} \\ Where n = the number of classes \\ TP = the number of true positive detections \\ FP = the number of false positive detections\end{matrix}$$

The following formula provides for AP used in the calculation of mAP.

$$\begin{matrix}A P = {\sum }_{k = 0}^{k = n - 1} [ Recalls ( k ) - Recalls ( k + 1 ) ] * Precisions ( k ) \\ Recalls ( n ) = 0 , Precisions ( n ) = 1 \\ n = Number of thresholds .\end{matrix}$$

The following formulas provide for precision and recall (used in the calculation of AP).

$$\begin{matrix}Precision = \frac{tp}{tp + fp} \\ Recall = \frac{tp}{tp + fn}\end{matrix}$$

Wherein TP means the number of true positive detections (per class), FP means the number of false positive detections (per class), and FN means the number of false negative detections (per class).

In an embodiment, identification, ranking and recognition of efficient surgeons may include the formula: (1+% unused/% used)*cost of all items, whereby items may include any surgical related items and/or procedure related items.

In an embodiment, improved efficiency ratio of sterile surgical items may include the formula: (1+% unused/% used)*cost of all items, whereby items may include any surgical related items and/or procedure related items.

### EXAMPLES

Practice of an aspect of an embodiment (or embodiments) of the invention will be still more fully understood from the following examples and experimental results, which are presented herein for illustration only and should not be construed as limiting the invention in any way.

### Example and Experimental Results Set No. 1

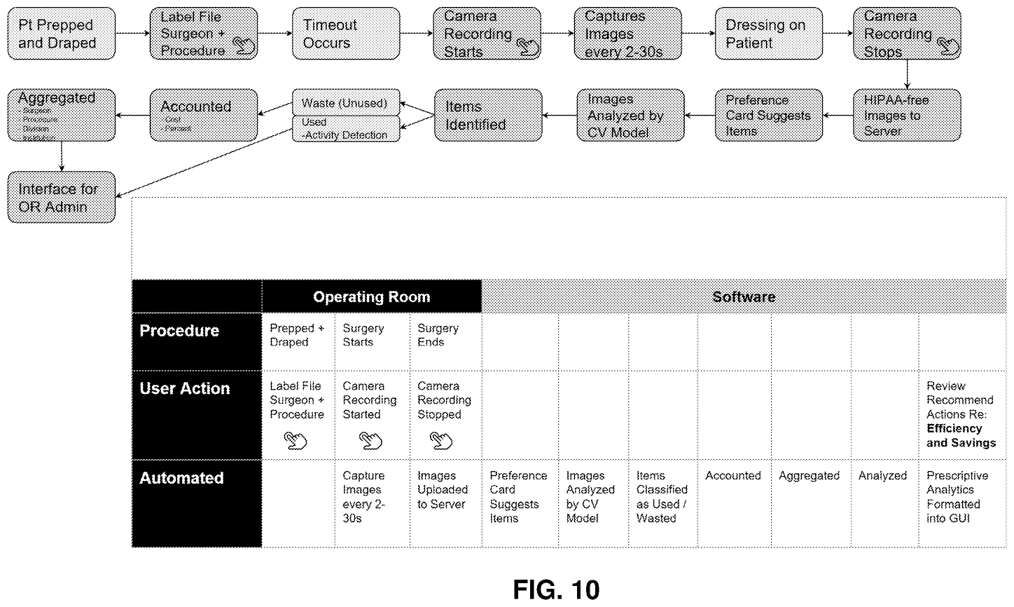

FIG. 9 is a screenshot of a flow diagram of a method for determining one or more characteristics of surgical related items and/or procedure related items present at preoperative, intraoperative, and/or postoperative settings and/or simulated preoperative, intraoperative, and/or postoperative settings, such as in the instant illustration providing a workflow for sterile surgical items (SSI). The method provides novel data including, but not limited thereto, the determination of unused items, unnecessarily used items, and clinically used items. The method can be performed by a system of one or more appropriately-programmed computers or processors in one or more locations. A list of acronyms present in the flow diagram are provided as follows: sterile surgical items (SSI), chief financial officer (CFO), chief medical officer (CMO), group purchasing organizations (GPO's), single-use, sterile surgical supplies (SUSSS), sterile surgical processing (SSP), and registered nurse (RN).

### Example and Experimental Results Set No. 2

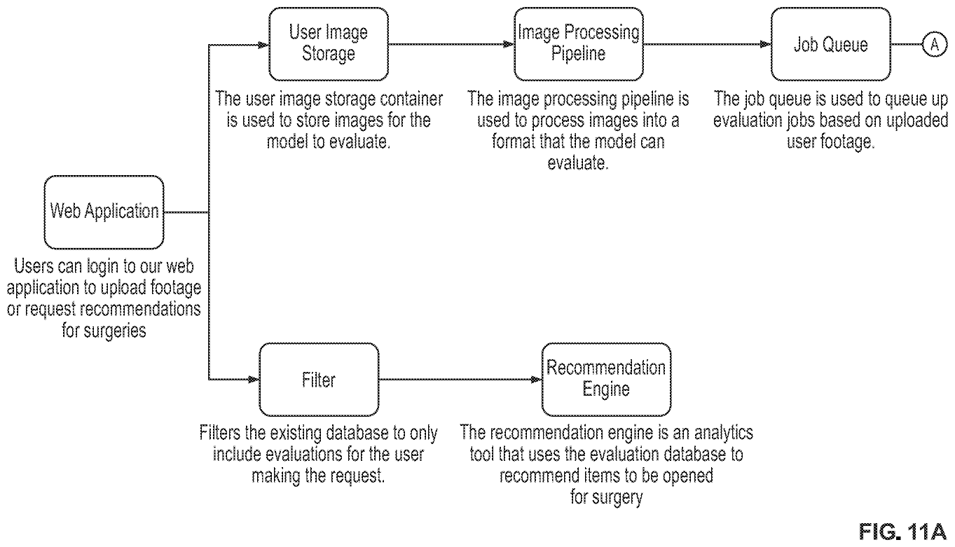

FIG. 10 is a screenshot of a flow diagram of a method and table for determining one or more characteristics of surgical related items and/or procedure related items present at preoperative, intraoperative, and/or postoperative settings and/or simulated preoperative, intraoperative, and/or postoperative settings, such as determining, but not limited thereto, unused waste items and activity of used items. The method can be performed by a system of one or more appropriately-programmed computers or processors in one or more locations.

### Example and Experimental Results Set No. 3

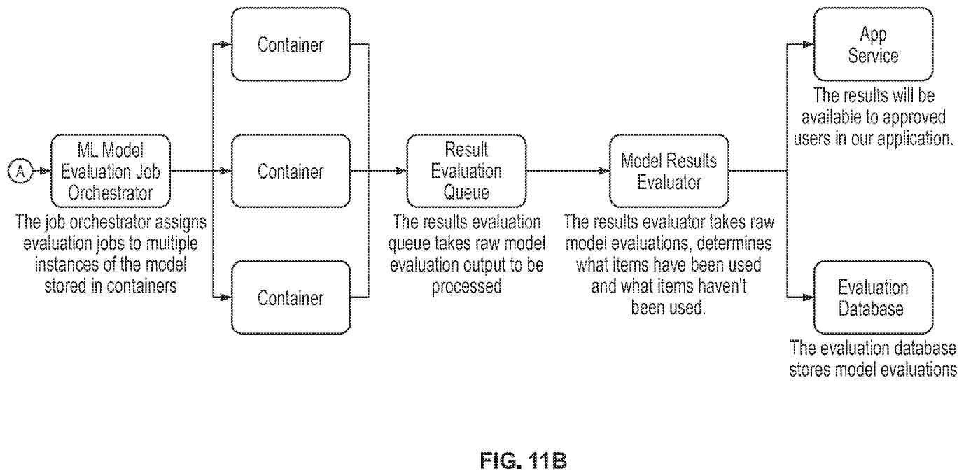

FIGS. 11(A)-(B) is a flow diagram of a method for determining one or more characteristics of surgical related items and/or procedure related items present at preoperative, intraoperative, and/or postoperative settings and/or simulated preoperative, intraoperative, and/or postoperative settings, such as determining, but not limited thereto, what items have been used and what items haven't been used and recommending, but not limited thereto, items to be opened for surgery. The method can be performed by a system of one or more appropriately-programmed computers or processors in one or more locations.

### Additional Examples

Example 1. A system configured for determining one or more characteristics of surgical related items and/or procedure related items present at preoperative, intraoperative, and/or postoperative settings and/or simulated preoperative, intraoperative, and/or postoperative settings. The system may comprise: one or more computer processors; and a memory configured to store instructions that are executable by said one or more computer processors. The one or more computer processors are configured to execute the instructions to: receive settings image data corresponding with the preoperative, intraoperative, and/or postoperative settings and/or simulated preoperative, intraoperative, and/or postoperative settings; run a trained computer vision model on the received settings image data to identify and label the surgical related items and/or procedure related items in the preoperative, intraoperative, and/or postoperative settings and/or simulated preoperative, intraoperative, and/or postoperative settings; interpret the surgical related items and/or procedure related items through tracking and analyzing said identified and labeled surgical related items and/or procedure related items in the preoperative, intraoperative, and/or postoperative settings and/or simulated preoperative, intraoperative, and/or postoperative settings, to determine said one or more characteristics of the surgical related items and/or procedure related items; and transmit said one or more determined characteristics to a secondary source.

Example 2. The system of example 1, wherein said one or more computer processors are configured to execute the instructions to: retrain said trained computer vision model using said received settings image data from the preoperative, intraoperative, and/or postoperative settings and/or simulated preoperative, intraoperative, and/or postoperative settings.

Example 3. The system of example 2, wherein said trained computer vision model is generated on preliminary image data using a machine learning algorithm.

Example 4. The system of example 3, wherein: said training of said computer vision model, may be performed with one or more of the following configurations:
i) streaming to the cloud and in real-time,
ii) streaming to the cloud and in delayed time,
iii) aggregated and delayed,
iv) locally on an edge-computing node, and
v) locally and/or remotely on a network and/or server.

Example 5. The system of example 2 (as well as subject matter of one or more of any combination of examples 3-4, in whole or in part), wherein: said retraining of said computer vision model, may be performed with one or more of the following configurations:
i) streaming to the cloud and in real-time,
ii) streaming to the cloud and in delayed time,
iii) aggregated and delayed,
iv) locally on an edge-computing node, and
v) locally and/or remotely on a network and/or server.

Example 6. The system of example 1 (as well as subject matter of one or more of any combination of examples 2-5, in whole or in part), wherein said trained computer vision model is generated on preliminary image data using a machine learning algorithm.

Example 7. The system of example 6, wherein: said training of said computer vision model, may be performed with one or more of the following configurations:
i) streaming to the cloud and in real-time,
ii) streaming to the cloud and in delayed time,
iii) aggregated and delayed,
iv) locally on an edge-computing node, and
v) locally and/or remotely on a network and/or server.

Example 8. The system of example 1 (as well as subject matter of one or more of any combination of examples 2-7, in whole or in part), wherein one or more of the following instructions:
a) said receiving of said settings image data,
b) said running of said trained computer vision model, and
c) said interpreting of the surgical related items and/or procedure related items,
may be performed with one or more of the following configurations:
i) streaming to the cloud and in real-time,
ii) streaming to the cloud and in delayed time,
iii) aggregated and delayed,
iv) locally on an edge-computing node, and
v) locally and/or remotely on a network and/or server.

Example 9. The system of example 1 (as well as subject matter of one or more of any combination of examples 2-8, in whole or in part), wherein said tracking and analyzing comprises one or more of the following:
object identification for tracking and analyzing;
motion sensing for tracking and analyzing;
depth and distance assessment for tracking and analyzing; and
infrared sensing for tracking and analyzing.

Example 10. The system of example 1 (as well as subject matter of one or more of any combination of examples 2-9, in whole or in part), wherein said tracking and analyzing comprises specified multiple tracking and analyzing models.

Example 11. The system of example 1 (as well as subject matter of one or more of any combination of examples 2-10, in whole or in part), wherein said one or more computer processors are configured to execute the instructions for said tracking and analyzing at one or more of the following:
one or more databases;
cloud infrastructure; and
edge-computing;

Example 12. The system of example 1 (as well as subject matter of one or more of any combination of examples 2-11, in whole or in part), wherein said secondary source includes one or more of any one of the following:
local memory;
remote memory; or
display or graphical user interface.

Example 13. The system of example 1 (as well as subject matter of one or more of any combination of examples 2-12, in whole or in part), wherein the machine learning algorithm includes an artificial neural network (ANN) or deep learning algorithm.

Example 14. The system of example 13, wherein said artificial neural network (ANN) includes:
convolutional neural network (CNN); and/or
recurrent neural networks (RNN).

Example 15. The system of example 1 (as well as subject matter of one or more of any combination of examples 2-14, in whole or in part), wherein said determined one or more characteristics includes any combination of one or more of the following:
identification of the one or more of the surgical related items and/or procedure related items;
usage or non-usage status of the one or more of the surgical related items and/or procedure related items;
opened or unopened status of the one or more of the surgical related items and/or procedure related items;
moved or non-moved status of the one or more of the surgical related items and/or procedure related items;
single-use or reusable status of the one or more of the surgical related items and/or procedure related items; or
association of clinical events, logistical events, or operational events.

Example 16. The system of example 1 (as well as subject matter of one or more of any combination of examples 2-15, in whole or in part), further comprising: one or more cameras configured to capture the image to provide said received image data.

Example 17. The system of example 1 (as well as subject matter of one or more of any combination of examples 2-16, in whole or in part), wherein said one or more computer processors are further configured to, based on said determined one or more characteristics, execute the instructions to:
determine an actionable output to reduce unnecessary waste of the surgical related items for use in the preoperative, intraoperative, and/or postoperative settings and/or the simulated preoperative, intraoperative phase, and/or postoperative settings;
determine an actionable output to reorganize the surgical related items for use in the preoperative, intraoperative, and/or postoperative settings or the simulated preoperative, intraoperative, and/or postoperative settings;
determine an actionable output to reduce supply, storage, sterilization and disposal costs associated with use of the surgical related items for use in the preoperative, intraoperative, and/or postoperative settings and/or the simulated preoperative, intraoperative, and/or postoperative settings;
determine an actionable output to reduce garbage and unnecessary re-sterilization associated with use of the surgical related items for use in the preoperative, intraoperative, and/or postoperative settings and/or the simulated preoperative, intraoperative, and/or postoperative settings;
determine an actionable output to streamline setup of the surgical related items for use in the preoperative, intraoperative, and/or postoperative settings and/or the simulated preoperative, intraoperative, and/or postoperative settings;
determine an actionable output to improve efficiency of using the surgical related items for use in the preoperative, intraoperative, and/or postoperative settings and/or the simulated preoperative, intraoperative, and/or postoperative settings;
determine an actionable output to identify, rank, and/or recognize level of efficiency of surgeons or clinicians; and/or
determine an actionable output to improve the level of efficiency of using the surgical related items and/or procedure related items that are sterilized.

Example 18. The system of example 1 (as well as subject matter of one or more of any combination of examples 2-17, in whole or in part), wherein neither machine readable markings on the surgical related items and/or procedure related items nor communicable coupling between said system and the surgical related items and/or procedure related items are required by said system to provide said one or more determined characteristics.

Example 19. The system of example 1 (as well as subject matter of one or more of any combination of examples 2-18, in whole or in part), wherein said settings image data comprises information from the visible light spectrum and/or invisible light spectrum.

Example 20. The system of example 1 (as well as subject matter of one or more of any combination of examples 2-19, in whole or in part), wherein said settings image data comprises three dimensional renderings or representation of information of the surgical related items and/or procedure related items in the preoperative, intraoperative, and/or postoperative settings and/or simulated preoperative, intraoperative, and/or postoperative settings.

Example 21. A computer-implemented method for determining one or more characteristics of surgical related items and/or procedure related items present at preoperative, intraoperative, and/or postoperative settings and/or simulated preoperative, intraoperative, and/or postoperative settings. The method may comprise: receiving settings image data corresponding with the preoperative, intraoperative, and/or postoperative settings and/or simulated preoperative, intraoperative, and/or postoperative settings; running a trained computer vision model on the received settings image data to identify and label the surgical related items and/or procedure related items in the preoperative, intraoperative, and/or postoperative settings and/or simulated preoperative, intraoperative, and/or postoperative settings; interpreting the surgical related items and/or procedure related items through tracking and analyzing said identified and labeled surgical related items and/or procedure related items in the preoperative, intraoperative, and/or postoperative settings and/or simulated preoperative, intraoperative, and/or postoperative settings, to determine said one or more characteristics of the surgical related items and/or procedure related items; and transmitting said one or more determined characteristics to a secondary source.

Example 22. The method of example 21, further comprising: retraining said trained computer vision model using said received settings image data from the preoperative, intraoperative, and/or postoperative settings and/or simulated preoperative, intraoperative, and/or postoperative settings.

Example 23. The method of example 22, wherein said trained computer vision model is generated on preliminary image data using a machine learning algorithm.

Example 24. The method of example 23, wherein: said training of said computer vision model, may be performed with one or more of the following configurations:
i) streaming to the cloud and in real-time,
ii) streaming to the cloud and in delayed time,
iii) aggregated and delayed,
iv) locally on an edge-computing node, and
v) locally and/or remotely on a network and/or server.

Example 25. The method of example 22 (as well as subject matter of one or more of any combination of examples 23-24, in whole or in part), wherein: said retraining of said computer vision model, may be performed with one or more of the following configurations:
i) streaming to the cloud and in real-time,
ii) streaming to the cloud and in delayed time,
iii) aggregated and delayed,
iv) locally on an edge-computing node, and
v) locally and/or remotely on a network and/or server.

Example 26. The method of example 21 (as well as subject matter of one or more of any combination of examples 22-25, in whole or in part), in whole or in part), wherein said trained computer vision model is generated on preliminary image data using a machine learning algorithm.

Example 27. The method of example 26, wherein: said training of said computer vision model, may be performed with one or more of the following configurations:
i) streaming to the cloud and in real-time,
ii) streaming to the cloud and in delayed time,
iii) aggregated and delayed,
iv) locally on an edge-computing node, and
v) locally and/or remotely on a network and/or server.

Example 28. The method of example 21 (as well as subject matter of one or more of any combination of examples 22-27), wherein one or more of the following actions:
a) said receiving of said settings image data,
b) said running of said trained computer vision model, and
c) said interpreting of the surgical related items and/or procedure related items,
may be performed with one or more of the following actions:
i) streaming to the cloud and in real-time,
ii) streaming to the cloud and in delayed time,
iii) aggregated and delayed,
iv) locally on an edge-computing node, and
v) locally and/or remotely on a network and/or server.

Example 29. The method of example 21 (as well as subject matter of one or more of any combination of examples 22-28), wherein said tracking and analyzing comprises one or more of the following:
object identification for tracking and analyzing;
motion sensing for tracking and analyzing;
depth and distance assessment for tracking and analyzing; and
infrared sensing for tracking and analyzing.

Example 30. The method of example 21 (as well as subject matter of one or more of any combination of examples 22-29), wherein said tracking and analyzing comprises specified multiple tracking and analyzing models.

Example 31. The method of example 21 (as well as subject matter of one or more of any combination of examples 22-30), wherein said tracking and analyzing may be performed with one or more of the following:
one or more databases;
cloud infrastructure; and
edge-computing;

Example 32. The method of example 21 (as well as subject matter of one or more of any combination of examples 22-31), wherein said secondary source includes one or more of any one of the following:
local memory;
remote memory; or
display or graphical user interface.

Example 33. The method of example 21 (as well as subject matter of one or more of any combination of examples 22-32), wherein the machine learning algorithm includes an artificial neural network (ANN) or deep learning algorithm.

Example 34. The method of example 33, wherein said artificial neural network (ANN) includes:
convolutional neural network (CNN); and/or
recurrent neural networks (RNN).

Example 35. The method of example 21 (as well as subject matter of one or more of any combination of examples 22-34), wherein said determined one or more characteristics includes any combination of one or more of the following: identification of the one or more of the surgical related items and/or procedure related items;
usage or non-usage status of the one or more of the surgical related items and/or procedure related items;
opened or unopened status of the one or more of the surgical related items and/or procedure related items;
moved or non-moved status of the one or more of the surgical related items and/or procedure related items;
single-use or reusable status of the one or more of the surgical related items and/or procedure related items; or
association of clinical events, logistical events, or operational events.

Example 36. The method of example 21 (as well as subject matter of one or more of any combination of examples 22-35), further comprising:
one or more cameras configured to capture the image to provide said received image data.

Example 37. The method of example 21 (as well as subject matter of one or more of any combination of examples 22-36), wherein based on said determined one or more characteristics, further comprising:
determining an actionable output to reduce unnecessary waste of the surgical related items for use in the preoperative, intraoperative, and/or postoperative settings and/or the simulated preoperative, intraoperative phase, and/or postoperative settings;
determining an actionable output to reorganize the surgical related items for use in the preoperative, intraoperative, and/or postoperative settings or the simulated preoperative, intraoperative, and/or postoperative settings;
determining an actionable output to reduce supply, storage, sterilization and disposal costs associated with use of the surgical related items for use in the preoperative, intraoperative, and/or postoperative settings and/or the simulated preoperative, intraoperative, and/or postoperative settings;
determining an actionable output to reduce garbage and unnecessary re-sterilization associated with use of the surgical related items for use in the preoperative, intraoperative, and/or postoperative settings and/or the simulated preoperative, intraoperative, and/or postoperative settings;
determining an actionable output to streamline setup of the surgical related items for use in the preoperative, intraoperative, and/or postoperative settings and/or the simulated preoperative, intraoperative, and/or postoperative settings;
determining an actionable output to improve efficiency of using the surgical related items for use in the preoperative, intraoperative, and/or postoperative settings and/or the simulated preoperative, intraoperative, and/or postoperative settings;
determining an actionable output to identify, rank, and/or recognize level of efficiency of surgeons or clinicians; and/or
determining an actionable output to improve the level of efficiency of using the surgical related items and/or procedure related items that are sterilized.

Example 38. The method of example 21 (as well as subject matter of one or more of any combination of examples 22-37), wherein neither machine readable markings on the surgical related items and/or procedure related items nor communicable coupling between said surgical related items and/or procedure related items and a system associated with said method are required by said method to provide said one or more determined characteristics.

Example 39. The method of example 21 (as well as subject matter of one or more of any combination of examples 22-38), wherein said settings image data comprises information from the visible light spectrum and/or invisible light spectrum.

Example 40. The method of example 21 (as well as subject matter of one or more of any combination of examples 22-39), wherein said settings image data comprises three dimensional renderings or representation of information of the surgical related items and/or procedure related items in the preoperative, intraoperative, and/or postoperative settings and/or simulated preoperative, intraoperative, and/or postoperative settings.

Example 41. A non-transitory computer readable medium storing instructions that, when executed by one or more processors, cause the one or more processors to perform operations for determining one or more characteristics of surgical related items and/or procedure related items present at preoperative, intraoperative, and/or postoperative settings and/or simulated preoperative, intraoperative, and/or postoperative settings. The non-transitory computer readable medium configured to cause the one or more processors to perform the following operations: receiving settings image data corresponding with the preoperative, intraoperative, and/or postoperative settings and/or simulated preoperative, intraoperative, and/or postoperative settings; running a trained computer vision model on the received settings image data to identify and label the surgical related items and/or procedure related items in the preoperative, intraoperative, and/or postoperative settings and/or simulated preoperative, intraoperative, and/or postoperative settings; interpreting the surgical related items and/or procedure related items through tracking and analyzing said identified and labeled surgical related items and/or procedure related items in the preoperative, intraoperative, and/or postoperative settings and/or simulated preoperative, intraoperative, and/or postoperative settings, to determine said one or more characteristics of the surgical related items and/or procedure related items; and transmitting said one or more determined characteristics to a secondary source.

Example 42. The non-transitory computer-readable medium of example 41, further comprising:
retraining said trained computer vision model using said received settings image data from the preoperative, intraoperative, and/or postoperative settings and/or simulated preoperative, intraoperative, and/or postoperative settings.

Example 43. The non-transitory computer-readable medium of example 42, wherein said rained computer vision model is generated on preliminary image data using a machine learning algorithm.

Example 44. The non-transitory computer-readable medium of example 43, wherein: said training of said computer vision model, may be performed with one or more of the following configurations:
i) streaming to the cloud and in real-time,
ii) streaming to the cloud and in delayed time,
iii) aggregated and delayed,
iv) locally on an edge-computing node, and
v) locally and/or remotely on a network and/or server.

Example 45. The non-transitory computer-readable medium of example 42 (as well as subject matter of one or more of any combination of examples 43-44, in whole or in part), wherein: said retraining of said computer vision model,
i) streaming to the cloud and in real-time,
ii) streaming to the cloud and in delayed time,
iii) aggregated and delayed,
iv) locally on an edge-computing node, and
v) locally and/or remotely on a network and/or server.

Example 46. The non-transitory computer-readable medium of example 41 (as well as subject matter of one or more of any combination of examples 42-45), wherein said trained computer vision model is generated on preliminary image data using a machine learning algorithm.

Example 47. The non-transitory computer-readable medium of example 46, wherein: said training of said computer vision model, may be performed with one or more of the following configurations:
i) streaming to the cloud and in real-time,
ii) streaming to the cloud and in delayed time,
iii) aggregated and delayed,
iv) locally on an edge-computing node, and
v) locally and/or remotely on a network and/or server.

Example 48. The non-transitory computer-readable medium of example 41 (as well as subject matter of one or more of any combination of examples 42-47), wherein one or more of the following actions:
a) said receiving of said settings image data,
b) said running of said trained computer vision model, and
c) said interpreting of the surgical related items and/or procedure related items,
may be performed with one or more of the following actions:
i) streaming to the cloud and in real-time,
ii) streaming to the cloud and in delayed time,
iii) aggregated and delayed,
iv) locally on an edge-computing node, and
v) locally and/or remotely on a network and/or server.

Example 49. The non-transitory computer-readable medium of example 41 (as well as subject matter of one or more of any combination of examples 42-48), wherein said tracking and analyzing comprises one or more of the following: object identification for tracking and analyzing;
motion sensing for tracking and analyzing;
depth and distance assessment for tracking and analyzing; and
infrared sensing for tracking and analyzing.

Example 50. The non-transitory computer-readable medium of example 41 (as well as subject matter of one or more of any combination of examples 42-49), wherein said tracking and analyzing comprises specified multiple tracking and analyzing models.

Example 51. The non-transitory computer-readable medium of example 41 (as well as subject matter of one or more of any combination of examples 42-50), wherein said tracking and analyzing may be configured to be performed with one or more of the following:
one or more databases;
cloud infrastructure; and
edge-computing;

Example 52. The non-transitory computer-readable medium of example 41 (as well as subject matter of one or more of any combination of examples 42-51), wherein said secondary source includes one or more of any one of the following:
local memory;
remote memory; or
display or graphical user interface.

Example 53. The non-transitory computer-readable medium of example 41 (as well as subject matter of one or more of any combination of examples 42-52), wherein the machine learning algorithm includes an artificial neural network (ANN) or deep learning algorithm.

Example 54. The non-transitory computer-readable medium of example 53, wherein said artificial neural network (ANN) includes:
convolutional neural network (CNN); and/or
recurrent neural networks (RNN).

Example 55. The non-transitory computer-readable medium of example 41 (as well as subject matter of one or more of any combination of examples 42-54), wherein said determined one or more characteristics includes any combination of one or more of the following:
identification of the one or more of the surgical related items and/or procedure related items;
usage or non-usage status of the one or more of the surgical related items and/or procedure related items;
opened or unopened status of the one or more of the surgical related items and/or procedure related items;
moved or non-moved status of the one or more of the surgical related items and/or procedure related items;
single-use or reusable status of the one or more of the surgical related items and/or procedure related items; or
association of clinical events, logistical events, or operational events.

Example 56. The non-transitory computer-readable medium of example 41 (as well as subject matter of one or more of any combination of examples 42-55), further comprising:
one or more cameras configured to capture the image to provide said received image data.

Example 57. The non-transitory computer-readable medium of example 41 (as well as subject matter of one or more of any combination of examples 42-56), wherein said one or more computer processors are further configured to, based on said determined one or more characteristics, execute the instructions to:
determine an actionable output to reduce unnecessary waste of the surgical related items for use in the preoperative, intraoperative, and/or postoperative settings and/or the simulated preoperative, intraoperative phase, and/or postoperative settings;
determine an actionable output to reorganize the surgical related items for use in the preoperative, intraoperative, and/or postoperative settings or the simulated preoperative, intraoperative, and/or postoperative settings;
determine an actionable output to reduce supply, storage, sterilization and disposal costs associated with use of the surgical related items for use in the preoperative, intraoperative, and/or postoperative settings and/or the simulated preoperative, intraoperative, and/or postoperative settings;
determine an actionable output to reduce garbage and unnecessary re-sterilization associated with use of the surgical related items for use in the preoperative, intraoperative, and/or postoperative settings and/or the simulated preoperative, intraoperative, and/or postoperative settings;
determine an actionable output to streamline setup of the surgical related items for use in the preoperative, intraoperative, and/or postoperative settings and/or the simulated preoperative, intraoperative, and/or postoperative settings;
determine an actionable output to improve efficiency of using the surgical related items for use in the preoperative, intraoperative, and/or postoperative settings and/or the simulated preoperative, intraoperative, and/or postoperative settings;
determine an actionable output to identify, rank, and/or recognize level of efficiency of surgeons or clinicians; and/or
determine an actionable output to improve the level of efficiency of using the surgical related items and/or procedure related items that are sterilized.

Example 58. The non-transitory computer-readable medium of example 41 (as well as subject matter of one or more of any combination of examples 42-57), wherein neither machine readable markings on the surgical related items and/or procedure related items nor communicable coupling between said surgical related items and/or procedure related items and a system associated with said computer readable medium are required by said system to provide said one or more determined characteristics.

Example 59. The non-transitory computer-readable medium of example 41 (as well as subject matter of one or more of any combination of examples 42-58), wherein said settings image data comprises information from the visible light spectrum and/or invisible light spectrum.

Example 60. The non-transitory computer-readable medium of example 41 (as well as subject matter of one or more of any combination of examples 42-59), wherein said settings image data comprises three dimensional renderings or representation of information of the surgical related items and/or procedure related items in the preoperative, intraoperative, and/or postoperative settings and/or simulated preoperative, intraoperative, and/or postoperative settings.

Example 61. A system configured to perform the method of any one or more of Examples 21-40, in whole or in part.

Example 62. A computer readable medium configured to perform the method of any one or more of Examples 21-40, in whole or in part.

Example 63. The method of using any of the elements, components, devices, computer readable medium, processors, memory, and/or systems, or their sub-components, provided in any one or more of examples 1-20, in whole or in part.

Example 64. The method of providing instructions to perform any one or more of Examples 21-40, in whole or in part.

Example 65. The method of manufacturing any of the elements, components, devices, computer readable medium, processors, memory, and/or systems, or their sub-components, provided in any one or more of examples 1-20, in whole or in part.

### REFERENCES

The devices, systems, apparatuses, modules, compositions, materials, compositions, computer program products, non-transitory computer readable medium, and methods of various embodiments of the invention disclosed herein may utilize aspects (such as devices, apparatuses, modules, systems, compositions, materials, compositions, computer program products, non-transitory computer readable medium, and methods) disclosed in the following references, applications, publications and patents and which are hereby incorporated by reference herein in their entirety (and which are not admitted to be prior art with respect to the present invention by inclusion in this section).
1. SHRANK et al., “Waste in the US Health Care System: Estimated Costs and Potential for Savings,” JAMA, Vol. 322, No. 15, Oct. 15, 2019 (Published online Oct. 7, 2019), pp. 1501-1509.
2. ZYGOURAKIS et al., “Operating Room Waste: Disposable Supply Utilization in Neurosurgical Procedures,” J Neurosurg, Vol. 126, February 2017 (Published online May 6, 2016), pp. 620-625.
3. CHEN et al., “iWaste: Video-Based Medical Waste Detection and Classification,” 2020 42^{nd} Annual International Conference of the IEEE Engineering in Medicine & Biology Society (EMBC), http://doi.org/10.1109/EMBC44109.2020.9175645, 14 pages.
4. U.S. Patent Application Publication No. US 2019/0206556 A1, Shelton, I V et al., “Real-Time Analysis of Comprehensive Cost of All Instrumentation Used in Surgery Utilizing Data Fluidity to Track Instruments through Stocking and In-House Processes”, Jul. 4, 2019.
5. U.S. Pat. No. 10,154,885 B1, Barnett, et al., “Systems, Apparatus and Methods for Continuously Tracking Medical Items throughout a Procedure”, Dec. 18, 2018.
6. IPAKTCHI et al., “Current Surgical Instrument Labeling Techniques May Increase the Risk of Unintentionally Retained Foreign Objects: A Hypothesis,” http://www.pssjournal.com/content/7/1/31, Patient Safety in Surgery, Vol. 7, 2013, 4 pages.
7. JAYADEVAN et al., “A Protocol to Recover Needles Lost During Minimally Invasive Surgery,” JSLS, Vol. 18, Issue 4, e2014.00165, October-December 2014, 6 pages.
8. BALLANGER, “Unique Device Identification of Surgical Instruments,” Feb. 5, 2017, pp. 1-23 (24 pages total).
9. LILLIS, “Identifying and Combatting Surgical Instrument Misuse and Abuse,” Infection Control Today, Nov. 6, 2015, 4 pages.
10. CHOBIN_{z} “Instrument-Marking Methods Must Be Maintained Properly,” Infection Control Today, Dec. 8, 2017, 2 pages.
11. LEE et al., “Automatic Surgical Instrument Recognition-A Case of Comparison Study between the Faster R-CNN, Mask R-CNN and Single-Shot Multi-Box Detectors,” Applied Sciences, Vol. 11, Aug. 31, 2021, pp. 1-17.
12. GILLMANN et al., “RFID for Medical Device and Surgical Instrument Tracking,” Medical Design Briefs, Sep. 1, 2018, 7 pages.
13. WYSS INSTITUTE, “Smart Tools: RFID Tracking for Surgical Instruments,” Smart Tools: RFID Tracking for Surgical Instruments (harvard.edu), 2022, 3 pages.
14. MURATA MANUFACTURING, “Surgical Tool Tracking with RFID,” Surgical tool tracking with RFID | Murata Manufacturing, 2022, 5 pages.
15. CENSIS, “What Are the Current Surgical Instrument Labeling Techniques,” What Are the Current Surgical Instrument Labeling Techniques? | Censis, 2022, 5 pages.
16. CENSIS, “CensiMark,” https://censis.com/solutions/censimark/, 2022, 2 pages.
17. Japanese Publication No. 2019-500921-A, “Systems and Methods for Data Capture in an Operating Room,” Jan. 17, 2019.
18. U.S. Publication No. 2016/007412-A1, DEIN, “Intra-Operative System for Identifying and Tracking Surgical Sharp Objects, Instruments, and Sponges,” Mar. 17, 2016.
19. U.S. Pat. No. 11,179,204-B2, SHELTON I V et al., “Wireless Pairing Of A Surgical Device With Another Device Within A Sterile Surgical Field Based On The Usage And Situational Awareness Of Devices,” Nov. 23, 2021.
20. U.S. Pat. No. 10,792,118-B2, PRPA et al., “Sterile Implant Tracking Device, System and Method of Use,” Oct. 6, 2020.
21. Australian Patent No. 2017216458-B2, HUMAYUN et al., “Sterile Surgical Tray,” Aug. 31, 2017.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the following claims including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

What is claimed is:

1. A system configured for determining one or more characteristics of surgical related items and/or procedure related items present at preoperative, intraoperative, and/or postoperative settings and/or simulated preoperative, intraoperative, and/or postoperative settings, comprising:
one or more computer processors;
a memory configured to store instructions that are executable by said one or more computer processors, wherein said one or more computer processors are configured to execute the instructions to:
receive settings image data corresponding with the preoperative, intraoperative, and/or postoperative settings and/or simulated preoperative, intraoperative, and/or postoperative settings;
run a trained computer vision model on the received settings image data to identify and label the surgical related items and/or procedure related items in the preoperative, intraoperative, and/or postoperative settings and/or simulated preoperative, intraoperative, and/or postoperative settings;
interpret the surgical related items and/or procedure related items through tracking and analyzing said identified and labeled surgical related items and/or procedure related items in the preoperative, intraoperative, and/or postoperative settings and/or simulated preoperative, intraoperative, and/or postoperative settings, to determine said one or more characteristics of the surgical related items and/or procedure related items; and
transmit said one or more determined characteristics to a secondary source.

2. The system of claim 1, wherein said one or more computer processors are configured to execute the instructions to:
retrain said trained computer vision model using said received settings image data from the preoperative, intraoperative, and/or postoperative settings and/or simulated preoperative, intraoperative, and/or postoperative settings.

3. The system of claim 2, wherein said trained computer vision model is generated on preliminary image data using a machine learning algorithm.

4. The system of claim 3, wherein: said training of said computer vision model, may be performed with one or more of the following configurations:
i) streaming to the cloud and in real-time,
ii) streaming to the cloud and in delayed time,
iii) aggregated and delayed,
iv) locally on an edge-computing node, and
v) locally and/or remotely on a network and/or server.

5. The system of claim 2, wherein: said retraining of said computer vision model, may be performed with one or more of the following configurations:
i) streaming to the cloud and in real-time,
ii) streaming to the cloud and in delayed time,
iii) aggregated and delayed,
iv) locally on an edge-computing node, and
v) locally and/or remotely on a network and/or server.

6. The system of claim 1, wherein said trained computer vision model is generated on preliminary image data using a machine learning algorithm.

7. The system of claim 6, wherein: said training of said computer vision model, may be performed with one or more of the following configurations:
i) streaming to the cloud and in real-time,
ii) streaming to the cloud and in delayed time,
iii) aggregated and delayed,
iv) locally on an edge-computing node, and
v) locally and/or remotely on a network and/or server.

8. The system of claim 1, wherein one or more of the following instructions:
a) said receiving of said settings image data,
b) said running of said trained computer vision model, and
c) said interpreting of the surgical related items and/or procedure related items,
may be performed with one or more of the following configurations:
i) streaming to the cloud and in real-time,
ii) streaming to the cloud and in delayed time,
iii) aggregated and delayed,
iv) locally on an edge-computing node, and
v) locally and/or remotely on a network and/or server.

9. The system of claim 1, wherein said tracking and analyzing comprises one or more of the following:
object identification for tracking and analyzing;
motion sensing for tracking and analyzing;
depth and distance assessment for tracking and analyzing; and
infrared sensing for tracking and analyzing.

10. The system of claim 1, wherein said tracking and analyzing comprises specified multiple tracking and analyzing models.

11. The system of claim 1, wherein said one or more computer processors are configured to execute the instructions for said tracking and analyzing at one or more of the following:
one or more databases;
cloud infrastructure; and
edge-computing.

12. The system of claim 1, wherein said secondary source includes one or more of any one of the following:
local memory;
remote memory; or
display or graphical user interface.

13. The system of claim 1, wherein the machine learning algorithm includes an artificial neural network (ANN) or deep learning algorithm.

14. The system of claim 13, wherein said artificial neural network (ANN) includes:
convolutional neural network (CNN); and/or
recurrent neural networks (RNN).

15. The system of claim 1, wherein said determined one or more characteristics includes any combination of one or more of the following:
identification of the one or more of the surgical related items and/or procedure related items;
usage or non-usage status of the one or more of the surgical related items and/or procedure related items;
opened or unopened status of the one or more of the surgical related items and/or procedure related items;
moved or non-moved status of the one or more of the surgical related items and/or procedure related items;
single-use or reusable status of the one or more of the surgical related items and/or procedure related items; or
association of clinical events, logistical events, or operational events.

16. The system of claim 1, further comprising:
one or more cameras configured to capture the image to provide said received image data.

17. The system of claim 1, wherein said one or more computer processors are further configured to, based on said determined one or more characteristics, execute the instructions to:
determine an actionable output to reduce unnecessary waste of the surgical related items for use in the preoperative, intraoperative, and/or postoperative settings and/or the simulated preoperative, intraoperative phase, and/or postoperative settings;
determine an actionable output to reorganize the surgical related items for use in the preoperative, intraoperative, and/or postoperative settings or the simulated preoperative, intraoperative, and/or postoperative settings;
determine an actionable output to reduce supply, storage, sterilization and disposal costs associated with use of the surgical related items for use in the preoperative, intraoperative, and/or postoperative settings and/or the simulated preoperative, intraoperative, and/or postoperative settings;
determine an actionable output to reduce garbage and unnecessary re-sterilization associated with use of the surgical related items for use in the preoperative, intraoperative, and/or postoperative settings and/or the simulated preoperative, intraoperative, and/or postoperative settings;
determine an actionable output to streamline setup of the surgical related items for use in the preoperative, intraoperative, and/or postoperative settings and/or the simulated preoperative, intraoperative, and/or postoperative settings;
determine an actionable output to improve efficiency of using the surgical related items for use in the preoperative, intraoperative, and/or postoperative settings and/or the simulated preoperative, intraoperative, and/or postoperative settings;
determine an actionable output to identify, rank, and/or recognize level of efficiency of surgeons or clinicians; and/or
determine an actionable output to improve the level of efficiency of using the surgical related items and/or procedure related items that are sterilized.

18. The system of claim 1, wherein neither machine readable markings on the surgical related items and/or procedure related items nor communicable coupling between said system and the surgical related items and/or procedure related items are required by said system to provide said one or more determined characteristics.

19. The system of claim 1, wherein said settings image data comprises information from the visible light spectrum and/or invisible light spectrum.

20. The system of claim 1, wherein said settings image data comprises three dimensional renderings or representation of information of the surgical related items and/or procedure related items in the preoperative, intraoperative, and/or postoperative settings and/or simulated preoperative, intraoperative, and/or postoperative settings.

21. A computer-implemented method for determining one or more characteristics of surgical related items and/or procedure related items present at preoperative, intraoperative, and/or postoperative settings and/or simulated preoperative, intraoperative, and/or postoperative settings, comprising:
receiving settings image data corresponding with the preoperative, intraoperative, and/or postoperative settings and/or simulated preoperative, intraoperative, and/or postoperative settings;
running a trained computer vision model on the received settings image data to identify and label the surgical related items and/or procedure related items in the preoperative, intraoperative, and/or postoperative settings and/or simulated preoperative, intraoperative, and/or postoperative settings;
interpreting the surgical related items and/or procedure related items through tracking and analyzing said identified and labeled surgical related items and/or procedure related items in the preoperative, intraoperative, and/or postoperative settings and/or simulated preoperative, intraoperative, and/or postoperative settings, to determine said one or more characteristics of the surgical related items and/or procedure related items; and
transmitting said one or more determined characteristics to a secondary source.

22. The method of claim 21, further comprising:
retraining said trained computer vision model using said received settings image data from the preoperative, intraoperative, and/or postoperative settings and/or simulated preoperative, intraoperative, and/or postoperative settings.

23. The method of claim 22, wherein said trained computer vision model is generated on preliminary image data using a machine learning algorithm.

24. The method of claim 23, wherein: said training of said computer vision model, may be performed with one or more of the following configurations:
i) streaming to the cloud and in real-time,
ii) streaming to the cloud and in delayed time,
iii) aggregated and delayed,
iv) locally on an edge-computing node, and
v) locally and/or remotely on a network and/or server.

25. The method of claim 22, wherein: said retraining of said computer vision model, may be performed with one or more of the following configurations:
i) streaming to the cloud and in real-time,
ii) streaming to the cloud and in delayed time,
iii) aggregated and delayed,
iv) locally on an edge-computing node, and
v) locally and/or remotely on a network and/or server.

26. The method of claim 21, wherein said trained computer vision model is generated on preliminary image data using a machine learning algorithm.

27. The method of claim 26, wherein: said training of said computer vision model, may be performed with one or more of the following configurations:
i) streaming to the cloud and in real-time,
ii) streaming to the cloud and in delayed time,
iii) aggregated and delayed,
iv) locally on an edge-computing node, and
v) locally and/or remotely on a network and/or server.

28. The method of claim 21, wherein one or more of the following actions:
a) said receiving of said settings image data,
b) said running of said trained computer vision model, and
c) said interpreting of the surgical related items and/or procedure related items,
may be performed with one or more of the following actions:
i) streaming to the cloud and in real-time,
ii) streaming to the cloud and in delayed time,
iii) aggregated and delayed,
iv) locally on an edge-computing node, and
v) locally and/or remotely on a network and/or server.

29. The method of claim 21, wherein said tracking and analyzing comprises one or more of the following:
object identification for tracking and analyzing;
motion sensing for tracking and analyzing;
depth and distance assessment for tracking and analyzing; and
infrared sensing for tracking and analyzing.

30. The method of claim 21, wherein said tracking and analyzing comprises specified multiple tracking and analyzing models.

31. The method of claim 21, wherein said tracking and analyzing may be performed with one or more of the following:
one or more databases;
cloud infrastructure; and
edge-computing.

32. The method of claim 21, wherein said secondary source includes one or more of any one of the following:
local memory;
remote memory; or
display or graphical user interface.

33. The method of claim 21, wherein the machine learning algorithm includes an artificial neural network (ANN) or deep learning algorithm.

34. The method of claim 33, wherein said artificial neural network (ANN) includes:
convolutional neural network (CNN); and/or
recurrent neural networks (RNN).

35. The method of claim 21, wherein said determined one or more characteristics includes any combination of one or more of the following:
identification of the one or more of the surgical related items and/or procedure related items;
usage or non-usage status of the one or more of the surgical related items and/or procedure related items;
opened or unopened status of the one or more of the surgical related items and/or procedure related items;
moved or non-moved status of the one or more of the surgical related items and/or procedure related items;
single-use or reusable status of the one or more of the surgical related items and/or procedure related items; or
association of clinical events, logistical events, or operational events.

36. The method of claim 21, further comprising:
one or more cameras configured to capture the image to provide said received image data.

37. The method of claim 21, wherein based on said determined one or more characteristics, further comprising:
determining an actionable output to reduce unnecessary waste of the surgical related items for use in the preoperative, intraoperative, and/or postoperative settings and/or the simulated preoperative, intraoperative phase, and/or postoperative settings;
determining an actionable output to reorganize the surgical related items for use in the preoperative, intraoperative, and/or postoperative settings or the simulated preoperative, intraoperative, and/or postoperative settings;
determining an actionable output to reduce supply, storage, sterilization and disposal costs associated with use of the surgical related items for use in the preoperative, intraoperative, and/or postoperative settings and/or the simulated preoperative, intraoperative, and/or postoperative settings;
determining an actionable output to reduce garbage and unnecessary re-sterilization associated with use of the surgical related items for use in the preoperative, intraoperative, and/or postoperative settings and/or the simulated preoperative, intraoperative, and/or postoperative settings;
determining an actionable output to streamline setup of the surgical related items for use in the preoperative, intraoperative, and/or postoperative settings and/or the simulated preoperative, intraoperative, and/or postoperative settings;
determining an actionable output to improve efficiency of using the surgical related items for use in the preoperative, intraoperative, and/or postoperative settings and/or the simulated preoperative, intraoperative, and/or postoperative settings;
determining an actionable output to identify, rank, and/or recognize level of efficiency of surgeons or clinicians; and/or
determining an actionable output to improve the level of efficiency of using the surgical related items and/or procedure related items that are sterilized.

38. The method of claim 21, wherein neither machine readable markings on the surgical related items and/or procedure related items nor communicable coupling between said surgical related items and/or procedure related items and a system associated with said method are required by said method to provide said one or more determined characteristics.

39. The method of claim 21, wherein said settings image data comprises information from the visible light spectrum and/or invisible light spectrum.

40. The method of claim 21, wherein said settings image data comprises three dimensional renderings or representation of information of the surgical related items and/or procedure related items in the preoperative, intraoperative, and/or postoperative settings and/or simulated preoperative, intraoperative, and/or postoperative settings.

41. A non-transitory computer readable medium storing instructions that, when executed by one or more processors, cause the one or more processors to perform operations for determining one or more characteristics of surgical related items and/or procedure related items present at preoperative, intraoperative, and/or postoperative settings and/or simulated preoperative, intraoperative, and/or postoperative settings, comprising:
receiving settings image data corresponding with the preoperative, intraoperative, and/or postoperative settings and/or simulated preoperative, intraoperative, and/or postoperative settings;
running a trained computer vision model on the received settings image data to identify and label the surgical related items and/or procedure related items in the preoperative, intraoperative, and/or postoperative settings and/or simulated preoperative, intraoperative, and/or postoperative settings;
interpreting the surgical related items and/or procedure related items through tracking and analyzing said identified and labeled surgical related items and/or procedure related items in the preoperative, intraoperative, and/or postoperative settings and/or simulated preoperative, intraoperative, and/or postoperative settings, to determine said one or more characteristics of the surgical related items and/or procedure related items; and
transmitting said one or more determined characteristics to a secondary source.

42. The non-transitory computer-readable medium of claim 41, further comprising:
training said trained computer vision model using said received settings image data from the preoperative, intraoperative, and/or postoperative settings and/or simulated preoperative, intraoperative, and/or postoperative settings.

43. The non-transitory computer-readable medium of claim 42, wherein said trained computer vision model is generated on preliminary image data using a machine learning algorithm.

44. The non-transitory computer-readable medium of claim 43, wherein:
said training of said computer vision model, may be performed with one or more of the following configurations:
i) streaming to the cloud and in real-time,
ii) streaming to the cloud and in delayed time,
iii) aggregated and delayed,
iv) locally on an edge-computing node, and
v) locally and/or remotely on a network and/or server.

45. The non-transitory computer-readable medium of claim 42, wherein:
said retraining of said computer vision model, may be performed with one or more of the following configurations:
i) streaming to the cloud and in real-time,
ii) streaming to the cloud and in delayed time,
iii) aggregated and delayed,
iv) locally on an edge-computing node, and
v) locally and/or remotely on a network and/or server.

46. The non-transitory computer-readable medium of claim 41, wherein said trained computer vision model is generated on preliminary image data using a machine learning algorithm.

47. The non-transitory computer-readable medium of claim 46, wherein:
said training of said computer vision model, may be performed with one or more of the following configurations:
i) streaming to the cloud and in real-time,
ii) streaming to the cloud and in delayed time,
iii) aggregated and delayed,
iv) locally on an edge-computing node, and
v) locally and/or remotely on a network and/or server.

48. The non-transitory computer-readable medium of claim 41, wherein one or more of the following actions:
a) said receiving of said settings image data,
b) said running of said trained computer vision model, and
c) said interpreting of the surgical related items and/or procedure related items,
may be performed with one or more of the following actions:
i) streaming to the cloud and in real-time,
ii) streaming to the cloud and in delayed time,
iii) aggregated and delayed,
iv) locally on an edge-computing node, and
v) locally and/or remotely on a network and/or server.

49. The non-transitory computer-readable medium of claim 41, wherein said tracking and analyzing comprises one or more of the following:
object identification for tracking and analyzing;
motion sensing for tracking and analyzing;
depth and distance assessment for tracking and analyzing; and
infrared sensing for tracking and analyzing.

50. The non-transitory computer-readable medium of claim 41, wherein said tracking and analyzing comprises specified multiple tracking and analyzing models.

51. The non-transitory computer-readable medium of claim 41, wherein said tracking and analyzing may be configured to be performed with one or more of the following:
one or more databases;
cloud infrastructure; and
edge-computing.

52. The non-transitory computer-readable medium of claim 41, wherein said secondary source includes one or more of any one of the following:
local memory;
remote memory; or
display or graphical user interface.

53. The non-transitory computer-readable medium of claim 41, wherein the machine learning algorithm includes an artificial neural network (ANN) or deep learning algorithm.

54. The non-transitory computer-readable medium of claim 53, wherein said artificial neural network (ANN) includes:
convolutional neural network (CNN); and/or
recurrent neural networks (RNN).

55. The non-transitory computer-readable medium of claim 41, wherein said determined one or more characteristics includes any combination of one or more of the following:
identification of the one or more of the surgical related items and/or procedure related items;
usage or non-usage status of the one or more of the surgical related items and/or procedure related items;
opened or unopened status of the one or more of the surgical related items and/or procedure related items;
moved or non-moved status of the one or more of the surgical related items and/or procedure related items;
single-use or reusable status of the one or more of the surgical related items and/or procedure related items; or
association of clinical events, logistical events, or operational events.

56. The non-transitory computer-readable medium of claim 41, further comprising:
one or more cameras configured to capture the image to provide said received image data.

57. The non-transitory computer-readable medium of claim 41, wherein said one or more computer processors are further configured to, based on said determined one or more characteristics, execute the instructions to:
determine an actionable output to reduce unnecessary waste of the surgical related items for use in the preoperative, intraoperative, and/or postoperative settings and/or the simulated preoperative, intraoperative phase, and/or postoperative settings;
determine an actionable output to reorganize the surgical related items for use in the preoperative, intraoperative, and/or postoperative settings or the simulated preoperative, intraoperative, and/or postoperative settings;
determine an actionable output to reduce supply, storage, sterilization and disposal costs associated with use of the surgical related items for use in the preoperative, intraoperative, and/or postoperative settings and/or the simulated preoperative, intraoperative, and/or postoperative settings;
determine an actionable output to reduce garbage and unnecessary re-sterilization associated with use of the surgical related items for use in the preoperative, intraoperative, and/or postoperative settings and/or the simulated preoperative, intraoperative, and/or postoperative settings;
determine an actionable output to streamline setup of the surgical related items for use in the preoperative, intraoperative, and/or postoperative settings and/or the simulated preoperative, intraoperative, and/or postoperative settings;
determine an actionable output to improve efficiency of using the surgical related items for use in the preoperative, intraoperative, and/or postoperative settings and/or the simulated preoperative, intraoperative, and/or postoperative settings;
determine an actionable output to identify, rank, and/or recognize level of efficiency of surgeons or clinicians; and/or
determine an actionable output to improve the level of efficiency of using the surgical related items and/or procedure related items that are sterilized.

58. The non-transitory computer-readable medium of claim 41, wherein neither machine readable markings on the surgical related items and/or procedure related items nor communicable coupling between said surgical related items and/or procedure related items and a system associated with said computer readable medium are required by said system to provide said one or more determined characteristics.

59. The non-transitory computer-readable medium of claim 41, wherein said settings image data comprises information from the visible light spectrum and/or invisible light spectrum.

60. The non-transitory computer-readable medium of claim 41, wherein said settings image data comprises three dimensional renderings or representation of information of the surgical related items and/or procedure related items in the preoperative, intraoperative, and/or postoperative settings and/or simulated preoperative, intraoperative, and/or postoperative settings.
